SEQUENCE LISTING (93 sequences)

SEQ ID NO: 1 - DNA, 44 bp, synthetic construct
  source 1..44: mol_type = other DNA
gaaggtgaccaagttcatgcttattgaaagggacagcgtgcaac

SEQ ID NO: 2 - DNA, 44 bp, synthetic construct
  source 1..44: mol_type = other DNA
gaaggtcggagtcaacggatttattgaaagggacagcgtgcaag

SEQ ID NO: 3 - DNA, 25 bp, synthetic construct
  source 1..25: mol_type = other DNA
tgtacgacatacgtcgtcgatggag

SEQ ID NO: 4 - DNA, 45 bp, synthetic construct
  source 1..45: mol_type = other DNA
gaaggtgaccaagttcatgcttgtakaasgaatcrgttatgcatt

SEQ ID NO: 5 - DNA, 45 bp, synthetic construct
  source 1..45: mol_type = other DNA
gaaggtcggagtcaacggatttgtakaasgaatcrgttatgcatg

SEQ ID NO: 6 - DNA, 23 bp, synthetic construct
  source 1..23: mol_type = other DNA
tgcatgatgycgaacagagagaa

SEQ ID NO: 7 - DNA, 50 bp, synthetic construct
  source 1..50: mol_type = other DNA
gaaggtgaccaagttcatgctaaatttactatgatatggaytgtaaaacg

SEQ ID NO: 8 - DNA, 51 bp, synthetic construct
  source 1..51: mol_type = other DNA
gaaggtcggagtcaacggattgaaatttactatgatatggaytgtaaaact

SEQ ID NO: 9 - DNA, 21 bp, synthetic construct
  source 1..21: mol_type = other DNA
attytaagsmaaaawggaatt

SEQ ID NO: 10 - DNA, 44 bp, synthetic construct
  source 1..44: mol_type = other DNA
gaaggtgaccaagttcatgctaayttacaaraatcatgcccaga

SEQ ID NO: 11 - DNA, 43 bp, synthetic construct
  source 1..43: mol_type = other DNA
gaaggtcggagtcaacggattayttacaaraatcatgcccagg

SEQ ID NO: 12 - DNA, 23 bp, synthetic construct
  source 1..23: mol_type = other DNA
tatgcrgcaacrtcaataggtaa

SEQ ID NO: 13 - DNA, 40 bp, synthetic construct
  source 1..40: mol_type = other DNA
gaaggtgaccaagttcatgctcggtggccaaacgcgtatc

SEQ ID NO: 14 - DNA, 40 bp, synthetic construct
  source 1..40: mol_type = other DNA
gaaggtcggagtcaacggattcggtggccaaacgcgtata

SEQ ID NO: 15 - DNA, 24 bp, synthetic construct
  source 1..24: mol_type = other DNA
atggacattgacgaggcttgaagt

SEQ ID NO: 16 - DNA, 51 bp, synthetic construct
  source 1..51: mol_type = other DNA
gaaggtgaccaagttcatgctcaaggtaatatagtgttcttaaaaaacatc

SEQ ID NO: 17 - DNA, 51 bp, synthetic construct
  source 1..51: mol_type = other DNA
gaaggtcggagtcaacggattcaaggtaatatagtgttcttaaaaaacatt

SEQ ID NO: 18 - DNA, 25 bp, synthetic construct
  source 1..25: mol_type = other DNA
acagtctgggtaaaacaattagcgt

SEQ ID NO: 19 - DNA, 45 bp, synthetic construct
  source 1..45: mol_type = other DNA
gaaggtgaccaagttcatgctgctaacaactcaaaccgtcctcca

SEQ ID NO: 20 - DNA, 44 bp, synthetic construct
  source 1..44: mol_type = other DNA
gaaggtcggagtcaacggattctaacaactcaaaccgtcctccg

SEQ ID NO: 21 - DNA, 28 bp, synthetic construct
  source 1..28: mol_type = other DNA
ctttgaaattcgtgtttttttcatttcc

SEQ ID NO: 22 - DNA, 44 bp, synthetic construct
  source 1..44: mol_type = other DNA
gaaggtgaccaagttcatgcttttgttttgcttgcatgtgtgtc

SEQ ID NO: 23 - DNA, 44 bp, synthetic construct
  source 1..44: mol_type = other DNA
gaaggtcggagtcaacggatttttgttttgcttgcatgtgtgtg

SEQ ID NO: 24 - DNA, 25 bp, synthetic construct
  source 1..25: mol_type = other DNA
caggagagcaacaacatgtgatcat

SEQ ID NO: 25 - DNA, 43 bp, synthetic construct
  source 1..43: mol_type = other DNA
gaaggtgaccaagttcatgctggttctttctrcatttgttccg

SEQ ID NO: 26 - DNA, 44 bp, synthetic construct
  source 1..44: mol_type = other DNA
gaaggtcggagtcaacggattgggttctttctrcatttgttcca

SEQ ID NO: 27 - DNA, 23 bp, synthetic construct
  source 1..23: mol_type = other DNA
aacatcgggagaacaacagaagc

SEQ ID NO: 28 - DNA, 45 bp, synthetic construct
  source 1..45: mol_type = other DNA
gaaggtgaccaagttcatgctkgcatgctaactccatctaaaagt

SEQ ID NO: 29 - DNA, 45 bp, synthetic construct
  source 1..45: mol_type = other DNA
gaaggtcggagtcaacggattkgcatgctaactccatctaaaaga

SEQ ID NO: 30 - DNA, 26 bp, synthetic construct
  source 1..26: mol_type = other DNA
tttctgggtattgaagtcatgaaatt

SEQ ID NO: 31 - DNA, 43 bp, synthetic construct
  source 1..43: mol_type = other DNA
gaaggtgaccaagttcatgctcactcacgccatgcagtggacc

SEQ ID NO: 32 - DNA, 43 bp, synthetic construct
  source 1..43: mol_type = other DNA
gaaggtcggagtcaacggattcactcacgccatgcagtggaca

SEQ ID NO: 33 - DNA, 24 bp, synthetic construct
  source 1..24: mol_type = other DNA
agctgtgtcttgtggcctcgargt

SEQ ID NO: 34 - DNA, 45 bp, synthetic construct
  source 1..45: mol_type = other DNA
gaaggtgaccaagttcatgctatagtgccaagcccaaaagtccgg

SEQ ID NO: 35 - DNA, 45 bp, synthetic construct
  source 1..45: mol_type = other DNA
gaaggtcggagtcaacggattatagtgccaagcccaaaagtccga

SEQ ID NO: 36 - DNA, 21 bp, synthetic construct
  source 1..21: mol_type = other DNA
atcccgytrtgcamccagaac

SEQ ID NO: 37 - DNA, 47 bp, synthetic construct
  source 1..47: mol_type = other DNA
gaaggtgaccaagttcatgctaaatattacctcagcattccttctca

SEQ ID NO: 38 - DNA, 48 bp, synthetic construct
  source 1..48: mol_type = other DNA
gaaggtcggagtcaacggattcaaatattacctcagcattccttctct

SEQ ID NO: 39 - DNA, 25 bp, synthetic construct
  source 1..25: mol_type = other DNA
caataattgccaaatttccatttca

SEQ ID NO: 40 - DNA, 46 bp, synthetic construct
  source 1..46: mol_type = other DNA
gaaggtgaccaagttcatgctgaatgcattaacttttagctcctga

SEQ ID NO: 41 - DNA, 45 bp, synthetic construct
  source 1..45: mol_type = other DNA
gaaggtcggagtcaacggattaatgcattaacttttagctcctgg

SEQ ID NO: 42 - DNA, 26 bp, synthetic construct
  source 1..26: mol_type = other DNA
ataagcmaactaatttaaaaaaggtc

SEQ ID NO: 43 - DNA, 48 bp, synthetic construct
  source 1..48: mol_type = other DNA
gaaggtgaccaagttcatgcttatttttgtccactgatcctatactag

SEQ ID NO: 44 - DNA, 48 bp, synthetic construct
  source 1..48: mol_type = other DNA
gaaggtcggagtcaacggatttatttttgtccactgatcctatactaa

SEQ ID NO: 45 - DNA, 22 bp, synthetic construct
  source 1..22: mol_type = other DNA
cttccgctttctctatgcttgt

SEQ ID NO: 46 - DNA, 43 bp, synthetic construct
  source 1..43: mol_type = other DNA
gaaggtgaccaagttcatgctgctttcacaracggatcccctg

SEQ ID NO: 47 - DNA, 43 bp, synthetic construct
  source 1..43: mol_type = other DNA
gaaggtcggagtcaacggattgctttcacaracggatccccta

SEQ ID NO: 48 - DNA, 25 bp, synthetic construct
  source 1..25: mol_type = other DNA
gggataggggatctcaagcgaagtt

SEQ ID NO: 49 - DNA, 47 bp, synthetic construct
  source 1..47: mol_type = other DNA
gaaggtgaccaagttcatgctttcagttccaaaggacagaaatgact

SEQ ID NO: 50 - DNA, 46 bp, synthetic construct
  source 1..46: mol_type = other DNA
gaaggtcggagtcaacggatttcagttccaaaggacagaaatgaca

SEQ ID NO: 51 - DNA, 25 bp, synthetic construct
  source 1..25: mol_type = other DNA
aaacttccgggtctaactttcctga

SEQ ID NO: 52 - DNA, 44 bp, synthetic construct
  source 1..44: mol_type = other DNA
gaaggtgaccaagttcatgcttctgatttgcaggattagggagc

SEQ ID NO: 53 - DNA, 45 bp, synthetic construct
  source 1..45: mol_type = other DNA
gaaggtcggagtcaacggattttctgatttgcaggattagggaga

SEQ ID NO: 54 - DNA, 26 bp, synthetic construct
  source 1..26: mol_type = other DNA
tgtcatgaaatgaaatcacacctgaa

SEQ ID NO: 55 - DNA, 46 bp, synthetic construct
  source 1..46: mol_type = other DNA
gaaggtgaccaagttcatgctgtcaaatctagccrtttggaactca

SEQ ID NO: 56 - DNA, 46 bp, synthetic construct
  source 1..46: mol_type = other DNA
gaaggtcggagtcaacggattgtcaaatctagccrtttggaactcg

SEQ ID NO: 57 - DNA, 24 bp, synthetic construct
  source 1..24: mol_type = other DNA
cctgytggtaggaaccgaggaaag

SEQ ID NO: 58 - DNA, 45 bp, synthetic construct
  source 1..45: mol_type = other DNA
gaaggtgaccaagttcatgctcgtactggagaagaaagatacsaa

SEQ ID NO: 59 - DNA, 45 bp, synthetic construct
  source 1..45: mol_type = other DNA
gaaggtcggagtcaacggattcgtactggagaagaaagatacsag

SEQ ID NO: 60 - DNA, 25 bp, synthetic construct
  source 1..25: mol_type = other DNA
cggatcaggacggagttatactgtt

SEQ ID NO: 61 - DNA, 44 bp, synthetic construct
  source 1..44: mol_type = other DNA
gaaggtgaccaagttcatgcttgtgcaccatcctccggcagtcg

SEQ ID NO: 62 - DNA, 44 bp, synthetic construct
  source 1..44: mol_type = other DNA
gaaggtcggagtcaacggatttgtgcaccatcctccggcagtca

SEQ ID NO: 63 - DNA, 26 bp, synthetic construct
  source 1..26: mol_type = other DNA
ccggaatgttgagagactgggcgtgg

SEQ ID NO: 64 - DNA, 42 bp, synthetic construct
  source 1..42: mol_type = other DNA
gaaggtgaccaagttcatgctcgacgagatctaaaccgtggc

SEQ ID NO: 65 - DNA, 43 bp, synthetic construct
  source 1..43: mol_type = other DNA
gaaggtcggagtcaacggattccgacgagatctaaaccgtggt

SEQ ID NO: 66 - DNA, 22 bp, synthetic construct
  source 1..22: mol_type = other DNA
gcttgatgagtgagaaccgggc

SEQ ID NO: 67 - DNA, 46 bp, synthetic construct
  source 1..46: mol_type = other DNA
gaaggtgaccaagttcatgctaaggttagcacaacatttycmaaag

SEQ ID NO: 68 - DNA, 46 bp, synthetic construct
  source 1..46: mol_type = other DNA
gaaggtcggagtcaacggattaaggttagcacaacatttycmaaaa

SEQ ID NO: 69 - DNA, 24 bp, synthetic construct
  source 1..24: mol_type = other DNA
agagctgaggascaccagtatttg

SEQ ID NO: 70 - DNA, 49 bp, synthetic construct
  source 1..49: mol_type = other DNA
gaaggtgaccaagttcatgctacctttaactgcagcacatatgtcatct

SEQ ID NO: 71 - DNA, 48 bp, synthetic construct
  source 1..48: mol_type = other DNA
gaaggtcggagtcaacggattcctttaactgcagcacatatgtcatcc

SEQ ID NO: 72 - DNA, 25 bp, synthetic construct
  source 1..25: mol_type = other DNA
grgccagatggatttccagctttat

SEQ ID NO: 73 - DNA, 46 bp, synthetic construct
  source 1..46: mol_type = other DNA
gaaggtgaccaagttcatgctyagtgtccactrcataagagttcat

SEQ ID NO: 74 - DNA, 46 bp, synthetic construct
  source 1..46: mol_type = other DNA
gaaggtcggagtcaacggattyagtgtccactrcataagagttcac

SEQ ID NO: 75 - DNA, 22 bp, synthetic construct
  source 1..22: mol_type = other DNA
ggcarctgatgccttatccaga

SEQ ID NO: 76 - DNA, 50 bp, synthetic construct
  source 1..50: mol_type = other DNA
gaaggtgaccaagttcatgctcatgtatgatcttttgattaggtttcatc

SEQ ID NO: 77 - DNA, 50 bp, synthetic construct
  source 1..50: mol_type = other DNA
gaaggtcggagtcaacggattcatgtatgatcttttgattaggtttcata

SEQ ID NO: 78 - DNA, 23 bp, synthetic construct
  source 1..23: mol_type = other DNA
cgcggagtttcaacayggtaata

SEQ ID NO: 79 - DNA, 43 bp, synthetic construct
  source 1..43: mol_type = other DNA
gaaggtgaccaagttcatgctcggtaactggcttcgcatagta

SEQ ID NO: 80 - DNA, 43 bp, synthetic construct
  source 1..43: mol_type = other DNA
gaaggtcggagtcaacggattcggtaactggcttcgcatagtt

SEQ ID NO: 81 - DNA, 25 bp, synthetic construct
  source 1..25: mol_type = other DNA
attttactgatgcaagatgctcyct

SEQ ID NO: 82 - DNA, 48 bp, synthetic construct
  source 1..48: mol_type = other DNA
gaaggtgaccaagttcatgctcgtaaggtgaataataaaagagggatg

SEQ ID NO: 83 - DNA, 49 bp, synthetic construct
  source 1..49: mol_type = other DNA
gaaggtcggagtcaacggattacgtaaggtgaataataaaagagggata

SEQ ID NO: 84 - DNA, 24 bp, synthetic construct
  source 1..24: mol_type = other DNA
ttccatctccaggacttcaatcaa

SEQ ID NO: 85 - DNA, 41 bp, synthetic construct
  source 1..41: mol_type = other DNA
gaaggtgaccaagttcatgcttttggcgtgggarcaaccct

SEQ ID NO: 86 - DNA, 39 bp, synthetic construct
  source 1..39: mol_type = other DNA
gaaggtcggagtcaacggatttggcgtgggarcaacccc

SEQ ID NO: 87 - DNA, 25 bp, synthetic construct
  source 1..25: mol_type = other DNA
ggtgtagaagttggcgtcccactcc

SEQ ID NO: 88 - DNA, 44 bp, synthetic construct
  source 1..44: mol_type = other DNA
gaaggtgaccaagttcatgctctggggtgtttctcttcatggca

SEQ ID NO: 89 - DNA, 44 bp, synthetic construct
  source 1..44: mol_type = other DNA
gaaggtcggagtcaacggattctggggtgtttctcttcatggcc

SEQ ID NO: 90 - DNA, 25 bp, synthetic construct
  source 1..25: mol_type = other DNA
agctgctgcgtcacacgtgaactac

SEQ ID NO: 91 - DNA, 43 bp, synthetic construct
  source 1..43: mol_type = other DNA
gaaggtgaccaagttcatgctatcttttttcaattcatgaact

SEQ ID NO: 92 - DNA, 43 bp, synthetic construct
  source 1..43: mol_type = other DNA
gaaggtcggagtcaacggattatcttttttcaattcatgaacc

SEQ ID NO: 93 - DNA, 24 bp, synthetic construct
  source 1..24: mol_type = other DNA
aatttrgaaaaatttcatagattt

(19) United States
(12) Patent Application Publication
(10) Pub. No.: US 2026/0139330 A1
(43) Pub. Date: May 21, 2026

(54) PRIMER COMBINATION FOR KASP MARKERS USED IN THE IDENTIFICATION OF ELYMUS SIBIRICUS GERMPLASM RESOURCES AND APPLICATION THEREOF

(71) Applicant: Southwest University of Science and Technology, Mianyang (CN)

(72) Inventors: SHIQIE BAI, Mianyang (CN); JIAJUN YAN, Mianyang (CN); XINRUI LI, Mianyang (CN); MING SUN, Mianyang (CN); JING LIU, Mianyang (CN); PENGFEI DU, Mianyang (CN); WENLONG GOU, Mianyang (CN); TING WANG, Mianyang (CN); CHANJUAN WU, Mianyang (CN)

(21) Appl. No.: 19/342,900

(22) Filed: Sep. 29, 2025

(30) Foreign Application Priority Data
Nov. 19, 2024 (CN) 202411650904.9

(51) Int. Cl. C12Q 1/6895; C12Q 1/6806; C12Q 1/6809; C12Q 1/6827; G01N 21/64

(52) U.S. Cl. CPC C12Q 1/6895; C12Q 1/6806; C12Q 1/6809; C12Q 1/6827; G01N 21/6428; C12Q 2600/156; G01N 2021/6439

(57) ABSTRACT

The present invention discloses a primer combination for KASP markers used in the identification of Elymus sibiricus germplasm resources and application thereof, belonging to the technical field of the biotechnology. The primer is designed to amplify 31 SNP sites. Nucleotide sequences of KASP-SNP primers are provided sequentially as SEQ ID NOs: 1-93. Using the primer, the genetic distance matrix and phylogenetic tree of Elymus sibiricus germplasm can be constructed to determine genetic relationships among individuals. It can be applied to screen materials required for the Elymus sibiricus research and utilization, such as core germplasm construction, breeding population screening and genetic diversity studies, etc. The method provided by present invention offers advantages of high throughput, high accuracy, low cost, simplicity of operation and savings in manpower and material resources, and thus has broad application prospects.

### CROSS-REFERENCE TO RELATED APPLICATIONS

This application claims priority to Chinese Patent Application No. 202411650904.9, filed on Nov. 19, 2024, which is hereby incorporated by reference in its entirety.

### TECHNICAL FIELD

The present application contains a sequence listing which has been filed electronically in xml format and is hereby incorporated by reference in its entirety. Besides, a copy of the sequence listing in XML file is submitted later, the XML copy is created on Jul. 16, 2025, is named “primer combination for KASP markers used in the identification of Elymus sibiricus germplasm resources AND APPLICATion thereof” and is 84,912 bytes in size.

### BACKGROUND

The present invention belongs to technical field of biotechnology, and particularly relates to primer combination for KASP markers used in the identification of Elymus sibiricus germplasm resources and application thereof.

Elymus sibiricus L., commonly known as Siberian wild rye, is a typical species of the genus Elymus L. in the tribe Triticeae of the family Poaceae. It is a perennial, allopolyploid grass (2n=4×=28, StStHH) widely distributed across the Eurasian continent and often serves as dominant or constructive species in meadow steppe communities. Elymus sibiricus is not only known for its high forage quality but also for its strong adaptability and disease resistance to harsh environmental conditions, including high altitudes, cold, drought, and saline-alkali soils. It is extensively used for the restoration of degraded grasslands and the establishment of high-yield artificial pastures in the Qinghai-Tibet Plateau and other high-altitude regions of western China. Additionally, it serves as an important gene pool for improvement of cereal crops as well as forage species. Due to its high seed production potential, Elymus sibiricus is among a few native grass species from Qinghai-Tibet Plateau that has achieved large-scale seed production and commercial utilization.

The wild resources of Elymus sibiricus are broadly distributed and found in diverse habitats, including alpine meadows, forest glades, shrublands, alpine valleys as well as riverine gravel beds at the elevations ranging from 1,500 to 4,900 meters. Influenced by varying environmental and climatic conditions, significant phenotypic as well as genetic differences exist among the wild populations. These differences provide a rich genetic reservoir and diverse selection basis for the development and utilization of Elymus sibiricus germplasm. Identification of the wild populations is a prerequisite for the conservation and utilization of these resources. Currently, molecular-level studies on the assessment and genetic diversity of Elymus sibiricus germplasm are mainly limited to the second-generation molecular markers such as AFLP, ISSR, SSR, and SRAP. These markers not only have complicated operation steps and can not be automated, but also take a long time and have high labor costs for large-scale sample testing. Therefore, there is an urgent need to establish an accurate, efficient and cost-effective method for identification of Elymus sibiricus germplasm, so as to advance its genetic resource conservation, research, and utilization.

Competitive Allele-Specific PCR (KASP) is a high-throughput and automated molecular marker technology developed based on SNP and Indel sites. It is based on differences at terminal sites of markers and uses dual-fluorescence detection to distinguish two genotypes at a single SNP site, enabling precise biallelic genotyping of target SNPs in genomic DNA samples. As a new-generation SNP genotyping technology, KASP offers high accuracy, strong site adaptability and suitability for large-scale sample testing. It demonstrates advantages in genetic stability, accuracy, specificity, flexibility, test cost as well as detection efficiency. The KASP is widely recognized as the mainstream SNP genotyping tool in international plant and animal breeding programs. KASP marker libraries have been successfully developed for crops such as wheat and rice, where they play significant roles in genetic and breeding research. However, there haven't been reported applications or developments of KASP molecular markers for Elymus sibiricus.

### SUMMARY

The purpose of the present invention is to disclose primer combination for KASP markers used in the identification of Elymus sibiricus germplasm resources and application thereof, aiming to provide effective genetic resources and molecular markers for genetic diversity research, core germplasm construction, breeding population selection, germplasm resources as well as the variety rights protection of Elymus sibiricus.

To achieve the above objective, the present invention discloses a primer combination for KASP markers used in identification of Elymus sibiricus germplasm resources in the first aspect, wherein the primer combination is used to amplify 31 SNP sites, and wherein the basic information of the 31 SNP sites is shown in Table 2.

In the second aspect, the present invention discloses a KASP-SNP primer combination for identification of Elymus sibiricus germplasm resources, wherein the primer combinations are listed in Sequence Listing 1 (SEQ ID NOs: 1-93), wherein each KASP-SNP primer set consists of a first forward primer, a second forward primer and a reverse primer, and enables genotyping of the corresponding SNP sites through KASP. Detailed information for each primer set is shown in Table 1.

Furthermore, in each KASP-SNP primer set, the 5′ ends of the specific regions of the first forward primer as well as the second forward primer are respectively linked to different universal fluorescent tag sequences. More specifically, the universal fluorescent tag sequences are selected from FAM and VIC.

TABLE 1
31 KASP-SNP Primer Combinations (SEQ ID NOs: 1-93) for the
Identification of Elymus sibiricus Germplasm

| SNP | Primer | |
|---|---|---|
| locus | Code | Primer Sequence(5′-3′) |
| Es_SNP01 | KASP- | F1: GAAGGTGACCAAGTTCATGCTTATTGAAAGGGACAG |
| | SNP1 | CGTGCAAC |
| | | F2: GAAGGTCGGAGTCAACGGATTTATTGAAAGGGACAG |
| | | CGTGCAAG |
| | | R: TGTACGACATACGTCGTCGATGGAG |
| Es_SNP02 | KASP- | F1: GAAGGTGACCAAGTTCATGCTTGTAKAASGAATCR |
| | SNP2 | GTTATGCATT |
| | | F2: GAAGGTCGGAGTCAACGGATTTGTAKAASGAATCRG |
| | | TTATGCATG |
| | | R: TGCATGATGYCGAACAGAGAGAA |
| Es_SNP03 | KASP- | F1: GAAGGTGACCAAGTTCATGCTAAATTTACTATGATA |
| | SNP3 | TGGAYTGTAAAACG |
| | | F2: GAAGGTCGGAGTCAACGGATTGAAATTTACTATGAT |
| | | ATGGAYTGTAAAACT |
| | | R: ATTYTAAGSMAAAAWGGAATT |
| Es_SNP04 | KASP- | F1: GAAGGTGACCAAGTTCATGCTAAYTTACAARAATCAT |
| | SNP4 | GCCCAGA |
| | | F2: GAAGGTCGGAGTCAACGGATTAYTTACAARAATCATG |
| | | CCCAGG |
| | | R: TATGCRGCAACRTCAATAGGTAA |
| Es_SNP05 | KASP- | F1: GAAGGTGACCAAGTTCATGCTCGGTGGCCAAACGCGT |
| | SNP5 | ATC |
| | | F2: GAAGGTCGGAGTCAACGGATTCGGTGGCCAAACGCGT |
| | | ATA |
| | | R: ATGGACATTGACGAGGCTTGAAGT |
| Es_SNP06 | KASP- | F1: GAAGGTGACCAAGTTCATGCTCAAGGTAATATAGTG |
| | SNP6 | TTCTTAAAAAACATC |
| | | F2: GAAGGTCGGAGTCAACGGATTCAAGGTAATATAGTG |
| | | TTCTTAAAAAACATT |
| | | R: ACAGTCTGGGTAAAACAATTAGCGT |
| Es_SNP07 | KASP- | F1: GAAGGTGACCAAGTTCATGCTGCTAACAACTCAAAC |
| | SNP7 | CGTCCTCCA |
| | | F2: GAAGGTCGGAGTCAACGGATTCTAACAACTCAAACCG |
| | | TCCTCCG |
| | | R: CTTTGAAATTCGTGTTTTTTTCATTTCC |
| Es_SNP08 | KASP- | F1: GAAGGTGACCAAGTTCATGCTTTTGTTTTGCTTGCATG |
| | SNP8 | TGTGTC |
| | | F2: GAAGGTCGGAGTCAACGGATTTTTGTTTTGCTTGCAT |
| | | GTGTGTG |
| | | R: CAGGAGAGCAACAACATGTGATCAT |
| Es_SNP09 | KASP- | F1: GAAGGTGACCAAGTTCATGCTGGTTCTTTCTRCATTTG |
| | SNP9 | TTCCG |
| | | F2: GAAGGTCGGAGTCAACGGATTGGGTTCTTTCTRCATTT |
| | | GTTCCA |
| | | R: AACATCGGGAGAACAACAGAAGC |
| Es_SNP10 | KASP- | F1: GAAGGTGACCAAGTTCATGCTKGCATGCTAACTCCA |
| | SNP10 | TCTAAAAGT |
| | | F2: GAAGGTCGGAGTCAACGGATTKGCATGCTAACTCCAT |
| | | CTAAAAGA |
| | | R: TTTCTGGGTATTGAAGTCATGAAATT |
| Es_SNP11 | KASP- | F1: GAAGGTGACCAAGTTCATGCTCACTCACGCCATGCA |
| | SNP11 | GTGGACC |
| | | F2: GAAGGTCGGAGTCAACGGATTCACTCACGCCATGCAG |
| | | TGGACA |
| | | R: AGCTGTGTCTTGTGGCCTCGARGT |
| Es_SNP12 | KASP- | F1: GAAGGTGACCAAGTTCATGCTATAGTGCCAAGCCCAA |
| | SNP12 | AAGTCCGG |
| | | F2: GAAGGTCGGAGTCAACGGATTATAGTGCCAAGCCCAA |
| | | AAGTCCGA |
| | | R: ATCCCGYTRTGCAMCCAGAAC |
| Es_SNP13 | KASP- | F1: GAAGGTGACCAAGTTCATGCTAAATATTACCTCAGCA |
| | SNP13 | TTCCTTCTCA |
| | | F2: GAAGGTCGGAGTCAACGGATTCAAATATTACCTCAG |
| | | CATTCCTTCTCT |
| | | R: CAATAATTGCCAAATTTCCATTTCA |
| Es_SNP14 | KASP- | F1: GAAGGTGACCAAGTTCATGCTGAATGCATTAACTTT |
| | SNP14 | TAGCTCCTGA |
| | | F2: GAAGGTCGGAGTCAACGGATTAATGCATTAACTTTTA |
| | | GCTCCTGG |
| | | R: ATAAGCMAACTAATTTAAAAAAGGTC |
| Es_SNP15 | KASP- | F1: GAAGGTGACCAAGTTCATGCTTATTTTTGTCCACTGA |
| | SNP15 | TCCTATACTAG |
| | | F2: GAAGGTCGGAGTCAACGGATTTATTTTTGTCCACTGAT |
| | | CCTATACTAA |
| | | R: CTTCCGCTTTCTCTATGCTTGT |
| Es_SNP19 | KASP- | F1: GAAGGTGACCAAGTTCATGCTGCTTTCACARACGGAT |
| | SNP16 | CCCCTG |
| | | F2: GAAGGTCGGAGTCAACGGATTGCTTTCACARACGGA |
| | | TCCCCTA |
| | | R: GGGATAGGGGATCTCAAGCGAAGTT |
| Es_SNP17 | KASP- | F1: GAAGGTGACCAAGTTCATGCTTTCAGTTCCAAAGGA |
| | SNP17 | CAGAAATGACT |
| | | F2: GAAGGTCGGAGTCAACGGATTTCAGTTCCAAAGGACA |
| | | GAAATGACA |
| | | R: AAACTTCCGGGTCTAACTTTCCTGA |
| Es_SNP18 | KASP- | F1: GAAGGTGACCAAGTTCATGCTTCTGATTTGCAGGAT |
| | SNP18 | TAGGGAGC |
| | | F2: GAAGGTCGGAGTCAACGGATTTTCTGATTTGCAGGA |
| | | TTAGGGAGA |
| | | R: TGTCATGAAATGAAATCACACCTGAA |
| Es_SNP19 | KASP- | F1: GAAGGTGACCAAGTTCATGCTGTCAAATCTAGCCRTT |
| | SNP19 | TGGAACTCA |
| | | F2: GAAGGTCGGAGTCAACGGATTGTCAAATCTAGCCRT |
| | | TTGGAACTCG |
| | | R: CCTGYTGGTAGGAACCGAGGAAAG |
| Es_SNP20 | KASP- | F1: GAAGGTGACCAAGTTCATGCTCGTACTGGAGAAGAA |
| | SNP20 | AGATACSAA |
| | | F2: GAAGGTCGGAGTCAACGGATTCGTACTGGAGAAGAA |
| | | AGATACSAG |
| | | R: CGGATCAGGACGGAGTTATACTGTT |
| Es_SNP21 | KASP- | F1: GAAGGTGACCAAGTTCATGCTTGTGCACCATCCTCCG |
| | SNP21 | GCAGTCG |
| | | F2: GAAGGTCGGAGTCAACGGATTTGTGCACCATCCTCC |
| | | GGCAGTCA |
| | | R: CCGGAATGTTGAGAGACTGGGCGTGG |
| Es_SNP22 | KASP- | F1: GAAGGTGACCAAGTTCATGCTCGACGAGATCTAAACC |
| | SNP22 | GTGGC |
| | | F2: GAAGGTCGGAGTCAACGGATTCCGACGAGATCTAAAC |
| | | CGTGGT |
| | | R: GCTTGATGAGTGAGAACCGGGC |
| Es_SNP23 | KASP- | F1: GAAGGTGACCAAGTTCATGCTAAGGTTAGCACAACAT |
| | SNP23 | TTYCMAAAG |
| | | F2: GAAGGTCGGAGTCAACGGATTAAGGTTAGCACAACA |
| | | TTTYCMAAAA |
| | | R: AGAGCTGAGGASCACCAGTATTTG |
| Es_SNP24 | KASP- | F1: GAAGGTGACCAAGTTCATGCTACCTTTAACTGCAGCA |
| | SNP24 | CATATGTCATCT |
| | | F2: GAAGGTCGGAGTCAACGGATTCCTTTAACTGCAGCAC |
| | | ATATGTCATCC |
| | | R: GRGCCAGATGGATTTCCAGCTTTAT |
| Es_SNP25 | KASP- | F1: GAAGGTGACCAAGTTCATGCTYAGTGTCCACTRCATA |
| | SNP25 | AGAGTTCAT |
| | | F2: GAAGGTCGGAGTCAACGGATTYAGTGTCCACTRCATA |
| | | AGAGTTCAC |
| | | R: GGCARCTGATGCCTTATCCAGA |
| Es_SNP26 | KASP- | F1: GAAGGTGACCAAGTTCATGCTCATGTATGATCTTTTG |
| | SNP26 | ATTAGGTTTCATC |
| | | F2: GAAGGTCGGAGTCAACGGATTCATGTATGATCTTTTG |
| | | ATTAGGTTTCATA |
| | | R: CGCGGAGTTTCAACAYGGTAATA |
| Es_SNP27 | KASP- | F1: GAAGGTGACCAAGTTCATGCTCGGTAACTGGCTTCGC |
| | SNP27 | ATAGTA |
| | | F2: GAAGGTCGGAGTCAACGGATTCGGTAACTGGCTTCGC |
| | | ATAGTT |
| | | R: ATTTTACTGATGCAAGATGCTCYCT |
| Es_SNP28 | KASP- | F1: GAAGGTGACCAAGTTCATGCTCGTAAGGTGAATAATA |
| | SNP28 | AAAGAGGGATG |
| | | F2: GAAGGTCGGAGTCAACGGATTACGTAAGGTGAATAAT |
| | | AAAAGAGGGATA |
| | | R: TTCCATCTCCAGGACTTCAATCAA |
| Es_SNP29 | KASP- | F1: GAAGGTGACCAAGTTCATGCTTTTGGCGTGGGARCAA |
| | SNP29 | CCCT |
| | | F2: GAAGGTCGGAGTCAACGGATTTGGCGTGGGARCAAC |
| | | CCC |
| | | R: GGTGTAGAAGTTGGCGTCCCACTCC |
| Es_SNP30 | KASP- | F1: GAAGGTGACCAAGTTCATGCTCTGGGGTGTTTCTCTTC |
| | SNP30 | ATGGCA |
| | | F2: GAAGGTCGGAGTCAACGGATTCTGGGGTGTTTCTCTT |
| | | CATGGCC |
| | | R: AGCTGCTGCGTCACACGTGAACTAC |
| Es_SNP31 | KASP- | F1: GAAGGTGACCAAGTTCATGCTATCTTTTTTCAATTCAT |
| | SNP31 | GAACT |
| | | F2: GAAGGTCGGAGTCAACGGATTATCTTTTTTCAATTCA |
| | | TGAACC |
| | | R: AATTTRGAAAAATTTCATAGATTT |

In the third aspect, the present invention provides a kit, which comprises the KASP-SNP primer combination described in the second aspect of the present invention.

As an optional method, each primer in the above kit is packaged separately.

As an optional method, the kit further comprises reagents for KASP.

In the fourth aspect, the present invention also provides the use of the KASP-SNP primer combination described in the second aspect of the present invention in any of the following aspects:
1) construction of a DNA fingerprint database of standard Elymus sibiricus germplasm resources,
2) construction of a phylogenetic tree of Elymus sibiricus germplasm resources, and
3) high-throughput identification of the Elymus sibiricus germplasm to be tested and its relationship with the standard Elymus sibiricus germplasm.

Among them, the standard Elymus sibiricus germplasm was selected from the 60 Elymus sibiricus germplasms in Qinghai-Tibet Plateau, Northeast China, Northwest China, North China, Russia and Mongolia, representing the main distribution areas of the wild Elymus sibiricus. The information of the standard Elymus sibiricus germplasm is shown in Table 3.

In the fifth aspect, the present invention also provides a DNA fingerprint database of the standard Elymus sibiricus germplasm resources, comprising the genotypes (Table 7) obtained by precise bi-allelic typing of the 31 SNP sites (Table 2) described in the first aspect for each standard Elymus sibiricus germplasm based on the KASP marker primer combination for identification of Elymus sibiricus germplasm resources described in first aspect of present invention. The standard Elymus sibiricus germplasm is selected from 60 Elymus sibiricus germplasms in the Qinghai-Tibet Plateau, Northeast China, Northwest China, North China, Russia and Mongolia, representing main distribution areas of wild Elymus sibiricus. The standard Elymus sibiricus germplasm information is shown in Table 3.

The genotype of each standard Elymus sibiricus germplasm based on the above 31 SNP sites is determined as follows:
1) extracting genomic DNA from each standard Elymus sibiricus germplasm,
2) using the genomic DNA of each standard Elymus sibiricus germplasm obtained in step 1) as a template to perform KASP using the KASP-SNP primer combination described in second aspect or the kit described in third aspect, thereby obtaining amplification products, and
3) analyzing fluorescence signals of the amplified products, and determining genotyping of 31 SNP sites in a certain standard Elymus sibiricus germplasm genome based on fluorescence signals of the amplified products obtained by each KASP-SNP primer set.

The methods for determining the genotype at each of the 31 SNP sites in a given standard Elymus sibiricus germplasm are as follows:

If the fluorescence signal corresponding to a given SNP site matches fluorescence color of tag sequence attached to the first forward primer of the primer set that amplifies this SNP site, the genotype of the standard Elymus sibiricus germplasm based on the SNP site is a homozygous type with the same base as the base at corresponding position of the reference genome of Elymus sibiricus Chuancao No. 2. If the fluorescence signal matches the fluorescent tag sequence attached to the second forward primer of the primer set that amplifies this SNP site, the genotype of the standard Elymus sibiricus germplasm based on the SNP site is a homozygous mutant base type different from the base at corresponding position of the reference genome of Elymus sibiricus Chuancao No. 2. If the fluorescence signal is a combination of colors of the tag sequences attached to both the first and second forward primers, genotype of the standard Elymus sibiricus germplasm based on SNP site is heterozygous, one base is the same as the base at corresponding position of reference genome of Elymus sibiricus Chuancao No. 2, and the other base is a mutant base different from the base at corresponding position of the reference genome of Elymus sibiricus Chuancao No. 2.

In sixth aspect, present invention provides a method for high-throughput identification of Elymus sibiricus germplasm or identification of its relationship with the standard Elymus sibiricus germplasm, comprising the following steps:
1) extracting genomic DNA from the Elymus sibiricus sample to be tested;
2) using the genomic DNA obtained in step 1) as a template to perform the KASP, and using the primer combination according to claim 2 or the kit according to claim 5, thereby obtaining amplification products;
3) analyzing fluorescence signal of the amplified product, and determining the genotype of the 1st SNP site to the 31th SNP site in the Elymus sibiricus genome to be tested based on the fluorescence signal of the amplified product obtained by each KASP-SNP primer set;
4) comparing genotype results at SNP sites 1 to 31 of the Elymus sibiricus genome to be tested obtained in step 3) with those of the standard Elymus sibiricus germplasm stored in DNA fingerprint database, wherein it is determined that Elymus sibiricus germplasm to be tested does not belong to standard Elymus sibiricus germplasm if the number of differing SNP sites between Elymus sibiricus germplasm to be tested as well as a standard Elymus sibiricus germplasm is two or more, and wherein it is determined that Elymus sibiricus germplasm to be tested belongs to standard Elymus sibiricus germplasm or is suspected to be standard Elymus sibiricus germplasm if the number of the differing SNP sites is zero or one;
5) determining the phylogenetic relationship between Elymus sibiricus germplasm to be tested and standard Elymus sibiricus germplasm if Elymus sibiricus germplasm to be tested does not correspond to any of standard Elymus sibiricus germplasms in above DNA fingerprint database. The specific method is as follows: Based on genotyping results of the Elymus sibiricus germplasm to be tested and those of standard germplasms in the DNA fingerprint database, a genetic distance matrix as well as a phylogenetic tree are constructed. The phylogenetic relationship between the germplasm to be tested and the standard germplasm is then determined based on calculated genetic distances and clustering results in the phylogenetic tree. The smaller the genetic distance between the Elymus sibiricus germplasm to be tested and a certain standard Elymus sibiricus, the closer the relationship is, and they are obviously clustered together in the phylogenetic tree. Conversely, the farther the relationship is.

In the present invention, the reaction procedures of KASP are as follows: pre-denaturation at 95° C. for 10 min; denaturation at 95° C. for 20 s, annealing and extension at 61° C. to 55° C. for 1 min, 10 cycles, and a decrease of 0.6° C. per cycle: denaturation at 95° C. for 20 s, annealing at 55° C. for 60 s, and 27 cycles. After the PCR reaction is completed, the data is read. If the typing is not sufficient, amplification is continued. The amplification procedure is denaturation at 95° C. for 20 s, annealing at 55° C. for 60 s, and the typing is checked every 3 cycles until the typing is obvious.

The beneficial effects of the present invention compared with the prior art are as follows:

The present invention provides a group of KASP-SNP molecular markers that can be used for identifying Elymus sibiricus resources and an application method thereof, which can identify Elymus sibiricus germplasm resources with the high throughput, accuracy, stability, rapidity, high efficiency and low cost.

The invention utilizes reference genome and large-scale variation dataset of Elymus sibiricus to develop a set of KASP-SNP primer combination exhibiting polymorphism within Elymus sibiricus. The KASP-SNP primer combination provided by the present invention can be used to identify Elymus sibiricus germplasm and varieties, offering significant application value in clarifying the genetic background and variety rights of Elymus sibiricus resources during agricultural production and variety breeding. Additionally, the primers can be used to determine the phylogenetic relationships among the Elymus sibiricus individuals, providing effective genetic resources and molecular markers for studies on genetic diversity, construction of core germplasm sets and selection of breeding populations. The present invention fills the gap in molecular research of Elymus sibiricus by introducing KASP-SNP molecular markers and solves the problems that the current AFLP, ISSR, SSR, SRAP and other markers used in the study of Elymus sibiricus are cumbersome and time-consuming, the number of markers is limited, the detection data integration is difficult, and the large-scale accurate detection cannot be achieved. It realizes complete automation of reading of detection data in the identification of Elymus sibiricus resources, improves accuracy of the results and the detection efficiency and avoids cross-contamination as well as the generation of false positives. The method provided by the invention has advantages of high throughput, high accuracy, low cost, simple operation, saving manpower and material resources, etc., and therefore has broad application prospects.KASP-SNP1.

### BRIEF DESCRIPTION OF THE DRAWINGS

FIG. 1A is a genotyping diagram of 60 Elymus sibiricus germplasm populations detected by the KASP-SNP1 molecular marker of the present invention;

FIG. 1B is a genotyping diagram of 60 Elymus sibiricus germplasm populations detected by the KASP-SNP2 molecular marker of the present invention;

FIG. 1C is a genotyping diagram of 60 Elymus sibiricus germplasm populations detected by the KASP-SNP4 molecular marker of the present invention;

FIG. 1D is a genotyping diagram of 60 Elymus sibiricus germplasm populations detected by the KASP-SNP5 molecular marker of the present invention;

FIG. 1E is a genotyping diagram of 60 Elymus sibiricus germplasm populations detected by the KASP-SNP6 molecular marker of the present invention;

FIG. 1F is a genotyping diagram of 60 Elymus sibiricus germplasm populations detected by the KASP-SNP7 molecular marker of the present invention;

FIG. 1G is a genotyping diagram of 60 Elymus sibiricus germplasm populations detected by the KASP-SNP8 molecular marker of the present invention;

FIG. 1H is a genotyping diagram of 60 Elymus sibiricus germplasm populations detected by the KASP-SNP9 molecular marker of the present invention;

FIG. 1I is a genotyping diagram of 60 Elymus sibiricus germplasm populations detected by the KASP-SNP10 molecular marker of the present invention;

FIG. 1J is a genotyping diagram of 60 Elymus sibiricus germplasm populations detected by the KASP-SNP11 molecular marker of the present invention;

FIG. 1K is a genotyping diagram of 60 Elymus sibiricus germplasm populations detected by the KASP-SNP12 molecular marker of the present invention;

FIG. 1L is a genotyping diagram of 60 Elymus sibiricus germplasm populations detected by the KASP-SNP13 molecular marker of the present invention;

FIG. 1M is a genotyping diagram of 60 Elymus sibiricus germplasm populations detected by the KASP-SNP15 molecular marker of the present invention;

FIG. 1N is a genotyping diagram of 60 Elymus sibiricus germplasm populations detected by the KASP-SNP17 molecular marker of the present invention;

FIG. 1O is a genotyping diagram of 60 Elymus sibiricus germplasm populations detected by the KASP-SNP19 molecular marker of the present invention;

FIG. 1P is a genotyping diagram of 60 Elymus sibiricus germplasm populations detected by the KASP-SNP21 molecular marker of the present invention;

FIG. 1Q is a genotyping diagram of 60 Elymus sibiricus germplasm populations detected by the KASP-SNP22 molecular marker of the present invention;

FIG. 1R is a genotyping diagram of 60 Elymus sibiricus germplasm populations detected by the KASP-SNP23 molecular marker of the present invention;

FIG. 1S is a genotyping diagram of 60 Elymus sibiricus germplasm populations detected by the KASP-SNP24 molecular marker of the present invention;

FIG. 1T is a genotyping diagram of 60 Elymus sibiricus germplasm populations detected by the KASP-SNP25 molecular marker of the present invention;

FIG. 1U is a genotyping diagram of 60 Elymus sibiricus germplasm populations detected by the KASP-SNP26 molecular marker of the present invention;

FIG. 1V is a genotyping diagram of 60 Elymus sibiricus germplasm populations detected by the KASP-SNP27 molecular marker of the present invention;

FIG. 1W is a genotyping diagram of 60 Elymus sibiricus germplasm populations detected by the KASP-SNP29 molecular marker of the present invention;

FIG. 1X is a genotyping diagram of 60 Elymus sibiricus germplasm populations detected by the KASP-SNP30 molecular marker of the present invention.

If it: □ indicates that the FAM fluorescence on horizontal axis accounts for a higher proportion, the sample is homozygous type 1; ▴ indicates that VIC/HEX fluorescence on the vertical axis accounts for a higher proportion, the sample is homozygous type 2; ● indicates that proportions of two fluorescence in middle area are equal, the sample is heterozygous; ★ indicates negative control (NTC); × indicates undetected samples.

FIG. 2 is a phylogenetic tree constructed based on genotyping results of KASP markers for 60 Elymus sibiricus germplasms.

### DESCRIPTION OF EMBODIMENTS

The technical solutions in embodiments of the invention will be described clearly and completely below in conjunction with drawings in the embodiments of the invention. Obviously, the embodiments are only parts of themselves, rather than all embodiments. Based on embodiments in the present invention, all other embodiments obtained by ordinary technicians in the technical field without creative work are within the scope of protection of the present invention.

The present invention is further described in detail below in conjunction with the specific implementation methods. The examples given are only for illustrating the present invention, not for limiting protection scope of the present invention. The examples can be used as a guide for ordinary technicians in the field to make further improvements, but not constitute a limitation on the present invention in any way.

The experimental methods in the following embodiments, unless otherwise specified, are all conventional methods and carried out according to the techniques or conditions described in literature in the technical field or product instructions. The materials, reagents, etc. used in following embodiments, unless otherwise specified, can all be obtained from commercial channels.

### Embodiment 1: SNP Sites and KASP Primer Combinations for Identifying Elymus Sibiricus Resources

The present invention is based on whole-genome resequencing data from 90 accessions of Elymus sibiricus collected from Qinghai-Tibet Plateau, Northeast, Northwest, North China and from Russia and Mongolia. These 90 accessions cover major natural distribution regions of wild Elymus sibiricus and can represent a wide range of its genetic diversity. The sequencing data from these accessions were aligned to the Elymus sibiricus reference genome Chuancao No. 2, which is assembled by the research group of the inventors of the present invention (available at: https://www.biorxiv.org/content/10.1101/2024.04.17.589894v1). As a result, a total of 80,148,422 high-quality SNPs were identified. Among these, 31 SNP sites were selected based on the criteria suitable for KASP primer design. The final 31 SNP sites are listed in Table 2.

TABLE 2
Basic information of 31 SNP loci

| | Chromosome | Chromosomal | Base Type at SNP locus | |
|---|---|---|---|---|
| SNP locus | of SNP locus | location | Ref | Alt |
| Es_SNP01 | Es1H | 151369718 | C | G |
| Es_SNP02 | Es1H | 368738668 | T | G |
| Es_SNP03 | Es1St | 4252112 | G | T |
| Es_SNP04 | Es1St | 4254262 | A | G |
| Es_SNP05 | Es2H | 27018962 | C | A |
| Es_SNP06 | Es2H | 189406968 | C | T |
| Es_SNP07 | Es2H | 332856077 | A | G |
| Es_SNP08 | Es2H | 442576323 | C | G |
| Es_SNP09 | Es2St | 373302575 | G | A |
| Es_SNP10 | Es3H | 18510385 | T | A |
| Es_SNP11 | Es3H | 44159550 | C | A |
| Es_SNP12 | Es3St | 68726104 | G | A |
| Es_SNP13 | Es3St | 193423410 | A | T |
| Es_SNP14 | Es4H | 1322297 | A | G |
| Es_SNP15 | Es4H | 56665546 | G | A |
| Es_SNP16 | Es4H | 147223205 | G | A |
| Es_SNP17 | Es4H | 203852028 | T | A |
| Es_SNP18 | Es4St | 8175058 | C | A |
| Es_SNP19 | Es4St | 359198064 | A | G |
| Es_SNP20 | Es5H | 76547989 | A | G |
| Es_SNP21 | Es5St | 67286670 | G | A |
| Es_SNP22 | Es5St | 108042483 | C | T |
| Es_SNP23 | Es5St | 429659992 | G | A |
| Es_SNP24 | Es6St | 306971115 | T | C |
| Es_SNP25 | Es6St | 381408141 | T | C |
| Es_SNP26 | Es7H | 46465420 | C | A |
| Es_SNP27 | Es7H | 131252857 | A | T |
| Es_SNP28 | Es7H | 312478825 | G | A |
| Es_SNP29 | Es7H | 436380001 | T | C |
| Es_SNP30 | Es7St | 20075622 | A | C |
| Es_SNP31 | Es7St | 122289419 | T | C |

The specific methods are as follows:

### 1. Core SNP Site Screening

Based on the whole genome resequencing data of 90 Elymus sibiricus germplasms from Qinghai-Tibet Plateau, Northeast China, Northwest China, North China, Russia and Mongolia, these resources covers the main distribution areas of wild Elymus sibiricus and represents a wide range of genetic diversity of Elymus sibiricus. A total of 80,148,422 high-quality SNPs were identified, which were aligned to the reference genome of Elymus sibiricus Chuancao No. 2 using the software BWA (Burrows-Wheeler Aligner), and high-quality SNPs were identified using GATK (Genome Analysis Toolkit). The screening principles are as follows:
1) Specificity: To ensure that the selected SNP markers have high specificity, 200 bp of sequence upstream and downstream of each SNP were extracted, totaling 401 bp. Then, BLAST (Basic Local Alignment Search Tool) was performed to retain those SNPs that could be uniquely aligned to the reference genome.
2) Uniform distribution and high quality: Based on premise that SNP markers are evenly distributed on the 14 chromosomes of Elymus sibiricus, SNPs with no missing genotype data were retained, and SNPs with a minor allele frequency (MAF) lower than 20% were eliminated.
3) Polymorphism information content (PIC): The PIC value of SNP site was calculated using a Perl script, and sites with a PIC value less than 0.35 were eliminated.
4) Hardy-Weinberg equilibrium: Use VCFtools software and set the parameters—max-missing 1—maf 0.2—hwe 0.01 to retain the sites with p-values greater than 0.01 in the Hardy-Weinberg test.
5) Uniqueness: Perl script was used to screen out SNP sites with no other site mutations within 100 bp before and after the marker. 52 core SNP sites were screened out.

### 4. KASP-SNP Primer Design

For the 52 core SNP sites screened, 200 bp sequences upstream and downstream of SNP were extracted to design and develop KASP primers. Two allele-specific primers and one universal primer were designed for each KASP target site. The KASP was performed using Bio-Rad's CFX Connect™ real-time system. Finally, 31 core SNP sites were successfully converted into KASP markers (Table 1). Each KASP marker primer consists of two forward primers F1 and F2 with the different terminal bases and a reverse primer R. The universal fluorescent tag sequence FAM-tail which is shown as GAAGGTGACCAAGTTCATGCT (SEQ ID NO.94), which was added to the 5′ end of forward primer F1, and the universal fluorescent tag sequence VIC-tail which is shown as GAAGGTCGGAGTCAACGGATT (SEQ ID NO.95), which was added to the 5′ end of forward primer F2.

### Embodiment 2: Verification of the KASP-SNP Primers Developed in Embodiment 1 and Construction of a DNA Fingerprint Database of Elymus sibiricus Germplasm Based on 31 SNP Sites

The basic information of the 60 Elymus sibiricus germplasms for testing in the present embodiment is shown in Table 3. The germplasms for testing were selected from Qinghai-Tibet Plateau, Northeast China, Northwest China, North China, Russia and Mongolia, representing the main distribution areas of wild Elymus sibiricus.

TABLE 3
Basic information of 60 Elymus sibiricus germplasms for testing

| | | | | | above sea | |
|---|---|---|---|---|---|---|
| NO. | Sample serial number | Origin | Longitude | Latitude | level(m) | Region |
| 1 | SAG-NM18031/NC-test1 | Inner | 120.754 | 46.311 | 548 | NC |
| | | Mongolia | | | | |
| 2 | SAG-NM18033/NC-test2 | Inner | 118.617 | 44.247 | 963 | NC |
| | | Mongolia | | | | |
| 3 | SAG-NM18034/NC-test3 | Inner | 116.127 | 43.269 | 1340 | NC |
| | | Mongolia | | | | |
| 4 | SAG-NM18041/NC-test4 | Inner | 115.947 | 42.657 | 1301 | NC |
| | | Mongolia | | | | |
| 5 | SAG-NM18042/NC-test5 | Inner | 116.705 | 42.014 | 1155 | NC |
| | | Mongolia | | | | |
| 6 | SAG-NM18044/NC-test6 | Inner | 117.449 | 43.632 | 1509 | NC |
| | | Mongolia | | | | |
| 7 | SAG-NM18050/NC-test7 | Inner | 111.676 | 41.223 | 1621 | NC |
| | | Mongolia | | | | |
| 8 | SAG-HB18001/NC-test8 | Hebei | 115.008 | 41.479 | 1393.7 | NC |
| 9 | SAG-HB18004/NC-test9 | Hebei | 115.356 | 41.548 | 1412.6 | NC |
| 10 | SAG-HB18007/NC-test10 | Hebei | 116.013 | 41.563 | 1490.2 | NC |
| 11 | SAG-HB18013/NC-test11 | Hebei | 115.843 | 41.329 | 1670.6 | NC |
| 12 | SAG-HB18010/NC-test12 | Hebei | 115.870 | 41.389 | 1487.3 | NC |
| 13 | SAG-NM18006 | Inner | 123.984 | 50.477 | 468 | NC |
| | | Mongolia | | | | |
| 14 | SAG-NM18037 | Inner | 116.225 | 42.533 | 1380 | NC |
| | | Mongolia | | | | |
| 15 | SAG-NM18047 | Inner | 111.726 | 41.262 | 1722 | NC |
| | | Mongolia | | | | |
| 16 | SAG-NM18001/NE-test1 | Inner | 120.203 | 50.263 | 583 | NE |
| | | Mongolia | | | | |
| 17 | SAG-NM18007/NE-test2 | Inner | 122.413 | 50.650 | 746 | NE |
| | | Mongolia | | | | |
| 18 | SAG-NM18014/NE-test3 | Inner | 123.498 | 48.127 | 223 | NE |
| | | Mongolia | | | | |
| 19 | SAG-NM18015/NE-test4 | Inner | 121.214 | 49.595 | 988.08 | NE |
| | | Mongolia | | | | |
| 20 | SAG-NM18016/NE-test5 | Inner | 121.249 | 49.613 | 812 | NE |
| | | Mongolia | | | | |
| 21 | SAG-NM18017/NE-test6 | Inner | 121.249 | 49.612 | 800 | NE |
| | | Mongolia | | | | |
| 22 | SAG-NM18018/NE-test7 | Inner | 121.389 | 49.585 | 714 | NE |
| | | Mongolia | | | | |
| 23 | SAG-NM18020/NE-test8 | Inner | 117.674 | 49.382 | 571 | NE |
| | | Mongolia | | | | |
| 24 | SAG-NM18021/NE-test9 | Inner | 119.931 | 48.165 | 839 | NE |
| | | Mongolia | | | | |
| 25 | SAG-NM18025/NE-test10 | Inner | 119.515 | 47.418 | 880 | NE |
| | | Mongolia | | | | |
| 26 | SAG-NM18026/NE-test11 | Inner | 119.905 | 48.059 | 817 | NE |
| | | Mongolia | | | | |
| 27 | SAG-NM18004 | Inner | 121.610 | 50.652 | 745 | NE |
| | | Mongolia | | | | |
| 28 | SAG-NM18009 | Inner | 124.569 | 48.475 | 189 | NE |
| | | Mongolia | | | | |
| 29 | PI598775 | Russia | 132.025 | 43.858 | 107 | NE |
| 30 | PI598777 | Russia | 134.809 | 50.858 | 895 | NE |
| 31 | SAG-XJ18001/NW-test1 | Xinjiang | 81.075 | 43.368 | 2237.4 | NW |
| 32 | SAG-XJ18005/NW-test2 | Xinjiang | 81.046 | 43.107 | 1837.1 | NW |
| 33 | SAG-XJ18010/NW-test3 | Xinjiang | 89.762 | 47.126 | 1168 | NW |
| 34 | SAG-XJ18012/NW-test4 | Xinjiang | 89.769 | 47.147 | 1138.8 | NW |
| 35 | SAG-XJ18013/NW-test5 | Xinjiang | 90.270 | 46.543 | 1168.5 | NW |
| 36 | SAG-XJ18017/NW-test6 | Xinjiang | 90.261 | 46.490 | 1064.1 | NW |
| 37 | SAG-XJ18021/NW-test7 | Xinjiang | 89.207 | 43.768 | 1432 | NW |
| 38 | SAG-XJ18023/NW-test8 | Xinjiang | 87.300 | 43.583 | 1464.6 | NW |
| 39 | SAG-XJ18028/NW-test9 | Xinjiang | 87.174 | 43.532 | 1689.4 | NW |
| 40 | SAG-XJ18030/NW-test10 | Xinjiang | 87.178 | 43.518 | 1921 | NW |
| 41 | SAG-XJ18003 | Xinjiang | 81.123 | 43.224 | 2038.2 | NW |
| | SAG-XJ18003 | | | | | |
| 42 | SAG-XJ18007 | Xinjiang | 81.185 | 43.151 | 1841.1 | NW |
| | SAG-XJ18007 | | | | | |
| 43 | PI598781 | Russia | 107.317 | 51.763 | 518 | NW |
| 44 | PI598789 | Russia | 113.529 | 52.056 | 869 | NW |
| 45 | PI610862 | Mongolia | 106.443 | 47.407 | 1692 | NW |
| 46 | SAG-GS18003/QTP-test1 | Gansu | 102.582 | 35.198 | 3080 | QTP |
| 47 | SAG-GS18017/QTP-test2 | Gansu | 103.290 | 34.893 | 3200 | QTP |
| 48 | SAG-SC18007/QTP-test3 | Sichuan | 103.355 | 33.612 | 2816 | QTP |
| 49 | SAG-SC18010/QTP-test4 | Sichuan | 103.337 | 33.655 | 2677 | QTP |
| 50 | SAG-SC18015/QTP-test5 | Sichuan | 102.005 | 32.856 | 3407 | QTP |
| 51 | SAG-XZ18001/QTP-test6 | Xizang | 98.725 | 29.736 | 3580 | QTP |
| 52 | SAG-XZ18007/QTP-test7 | Xizang | 29.880 | 92.701 | 3969 | QTP |
| 53 | SAG-XZ18012/QTP-test8 | Xizang | 31.156 | 96.196 | 4108 | QTP |
| 54 | SAG-XZ18016/QTP-test9 | Xizang | 31.477 | 97.203 | 3340 | QTP |
| 55 | SAG-QH18010/QTP-test10 | Qinghai | 98.463 | 35.618 | 3780 | QTP |
| 56 | SAG-QH18015/QTP-test11 | Qinghai | 100.623 | 37.094 | 3310 | QTP |
| 57 | SAG-GS18011 | Gansu | 102.679 | 34.496 | 3010 | QTP |
| 58 | SAG-SC18001 | Sichuan | 102.620 | 31.880 | 3107.54 | QTP |
| 59 | SAG-SC18020 | Sichuan | 100.298 | 30.317 | 3563 | QTP |
| 60 | SAG-QH18002 | Qinghai | 102.046 | 36.224 | 2990 | QTP |

means: QTP: the population of Qinghai-Tibet Plateau; NE: the population of the Northeast; NW: the population of the Northwest; NC: the population of North China; XJ: the population of Xinjiang; GS: the population of Gansu; SC: the population of Sichuan; QH: the population of Qinghai; XZ: the population of Xizang; NM: the population of Neimeng; HB: the population of Hebei.

1. Extraction of Genomic DNA from Elymus sibiricus Germplasm for Testing

The genomic DNA of the Elymus sibiricus germplasm for testing was extracted using a plant DNA extraction kit (DP350, Tiangen Biochemical Technology (Beijing) Co., Ltd.), and the DNA concentration as well as purity were detected using NanoDrop2000 UV spectrophotometer (Thermo Fisher Scientific, MA, USA).

### 2. KASP Marker Verification

1) Newly synthesized primers were diluted to 10 μM with TE (pH 8.0), and then mixed with forward typing primer F1, forward typing primer F2 and downstream universal primer in a ratio of 1:1:3 and loaded onto the machine. 1.25 uL of the primer mixture was added to every 5 μL reaction system.
2) Dilution and addition of DNA samples: The genomic DNA samples of Elymus sibiricus germplasm for testing were diluted to a single-digit ratio according to lowest concentration of the sample, and the entire batch of samples was diluted. Each 5 μL reaction system contained 1.25 μL of the diluted DNA sample.
3) The construction of PCR reaction system (96-well plate) is shown in Table 4.

TABLE 4
PCR Reaction System

| reagent | 5 μL Reaction System | |
|---|---|---|
| 2* KASP master mix | 2.5 | μL |
| mixed primers | 1.25 | μL |
| DNA template | 1.25 | μL |
| water | 0 | μL |
| total | 5 | μL |

4) The 96-well PCR reaction plate was sealed, shaken, and centrifuged to ensure that the reaction system was evenly mixed.
5) After centrifugation, PCR amplification was performed. The amplification procedure is shown in Table 5.

### Table 5: PCR Amplification Procedure

TABLE 5
Amplification Program of PCR

| steps | process | temperature | | time | | cycle number |
|---|---|---|---|---|---|---|
| 1 | activation | 95° | C. | 10 | min | 1 |
| 2 | denaturation | 95° | C. | 20 | sec | 10 |
| | annealing/extension | 61-55° | C. | 60 | sec | |
| 3 | denaturation | 95° | C. | 20 | sec | 27 |
| | annealing/extension | 55° | C. | 60 | sec | |
| 4 | read | 25° | C. | 30 | sec | 1 |

6) If the typing result is not ideal, amplification will continue. The amplification program is shown in Table 6. The typing situation will be checked every 3 cycles until typing is obvious.

TABLE 6
Reaction Conditions of PCR

| steps | temperature | time | cycle number |
|---|---|---|---|
| 1 | 95° C. | 20 sec | 3 |
| | 55° C. | 60 sec | |

7) After completing the above steps, when temperature of each PCR amplification product drops below 25° C., the fluorescence value is read by scanning FAM and VIC beams of microplate reader. The present invention uses Omega Fluorostar scanner (BMG Labtech, Ortenberg, Germany) to detect the signals of the two fluorescent groups FAM and VIC, and uses Kluster Caller software (LGC Genomics, Beverly, MA, USA) to analyze the genotyping data.

Among the above 60 Elymus sibiricus germplasms for testing, genotype of each germplasm at each of the above 31 SNP sites constitutes the DNA fingerprint database of the standard Elymus sibiricus germplasm resource based on 31 SNP sites of the present invention (Table 7). The present database can be used to identify whether a certain Elymus sibiricus germplasm under an unknown genetic background belongs to above the 60 Elymus sibiricus germplasms for testing or to which specific germplasm it belongs.

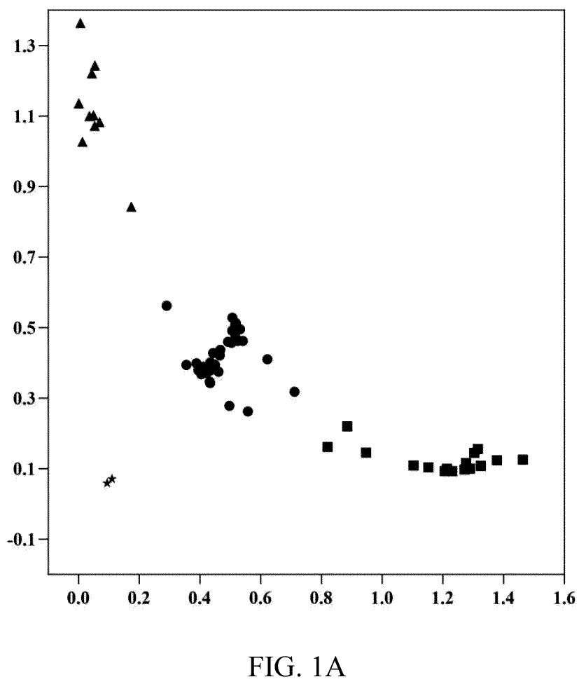

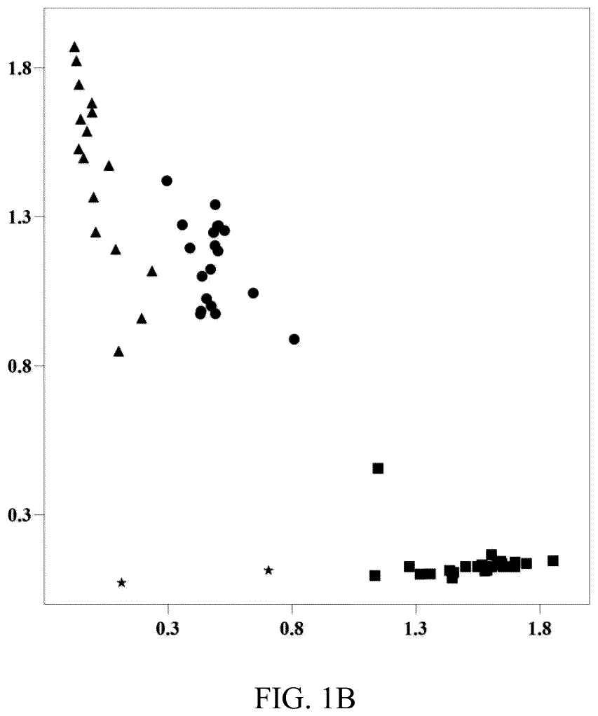

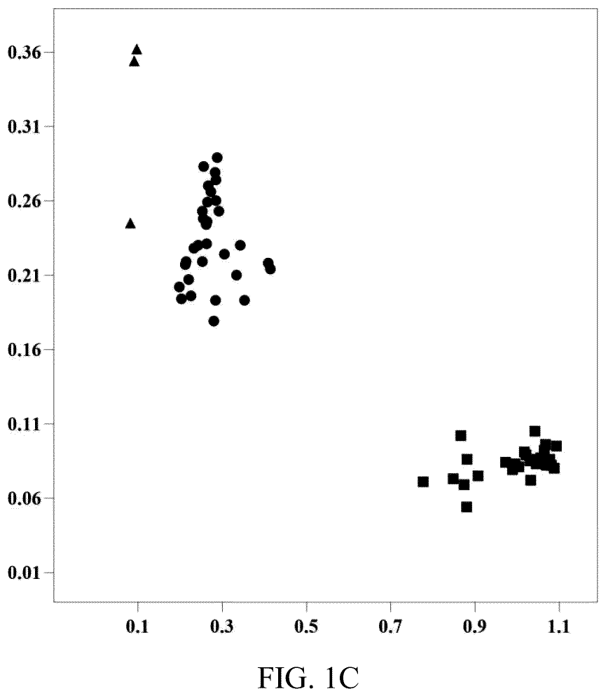

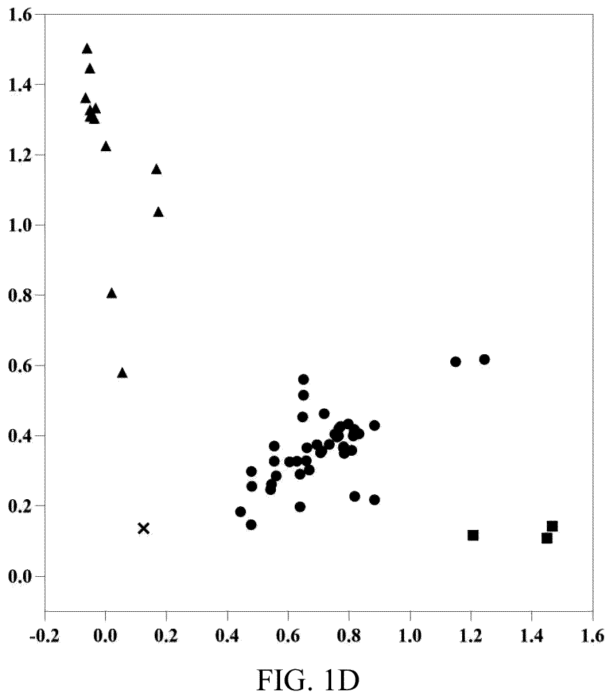

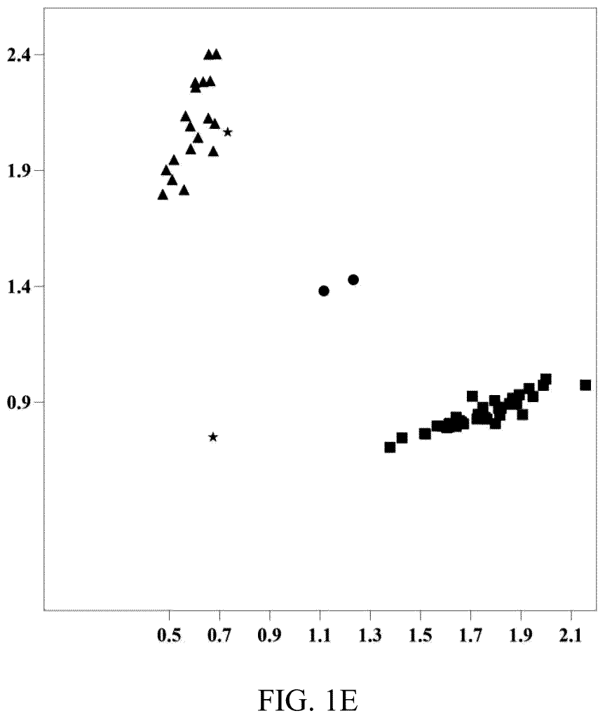

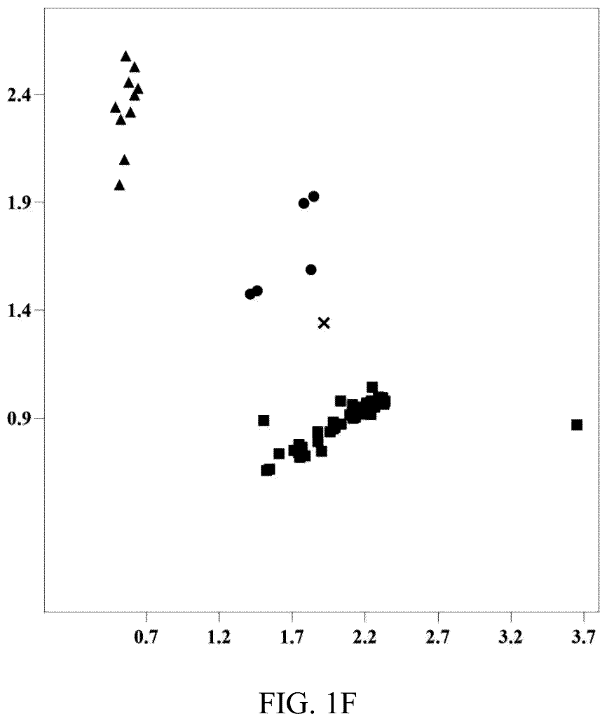

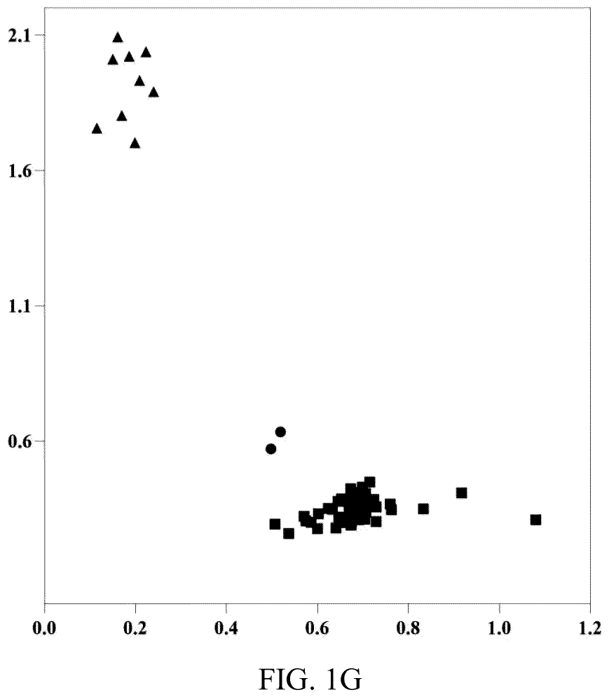

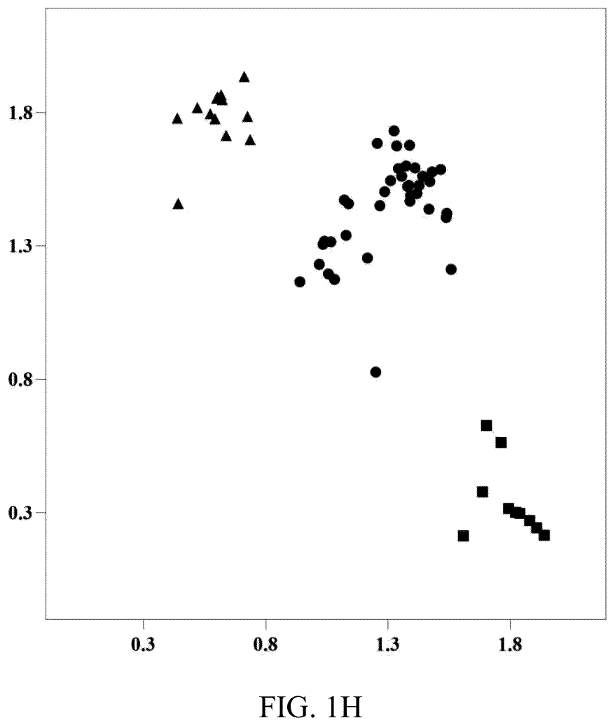

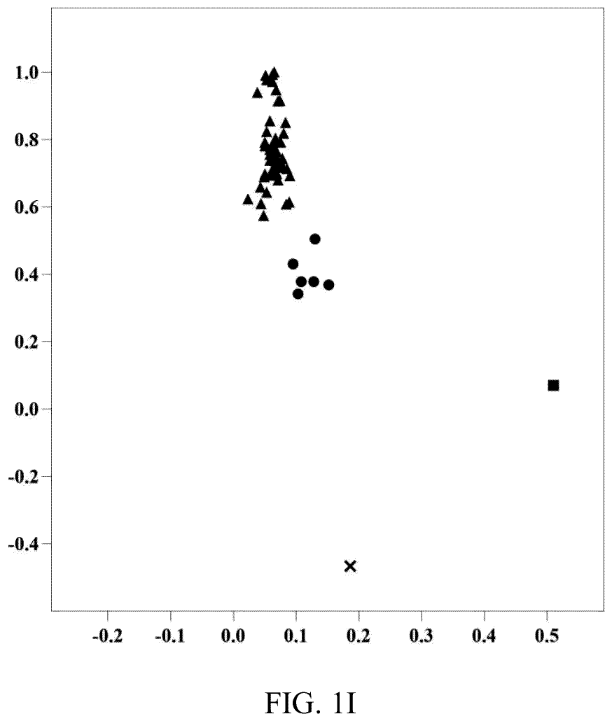

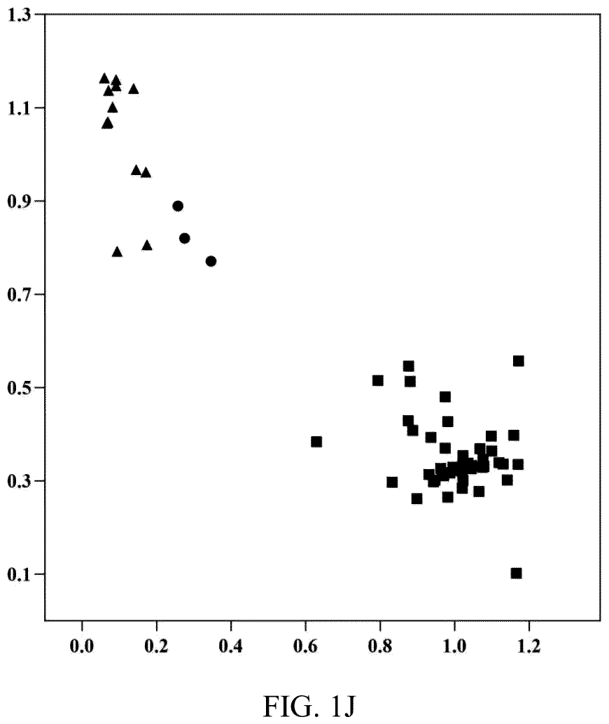

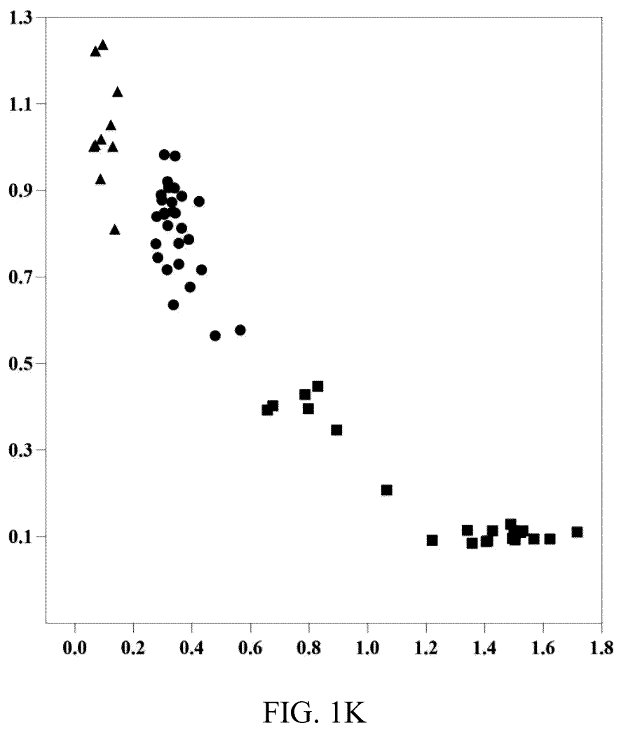

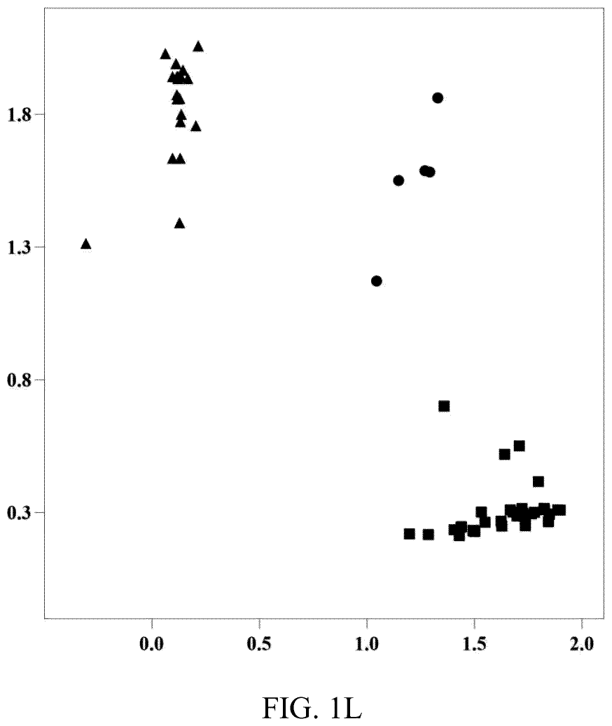

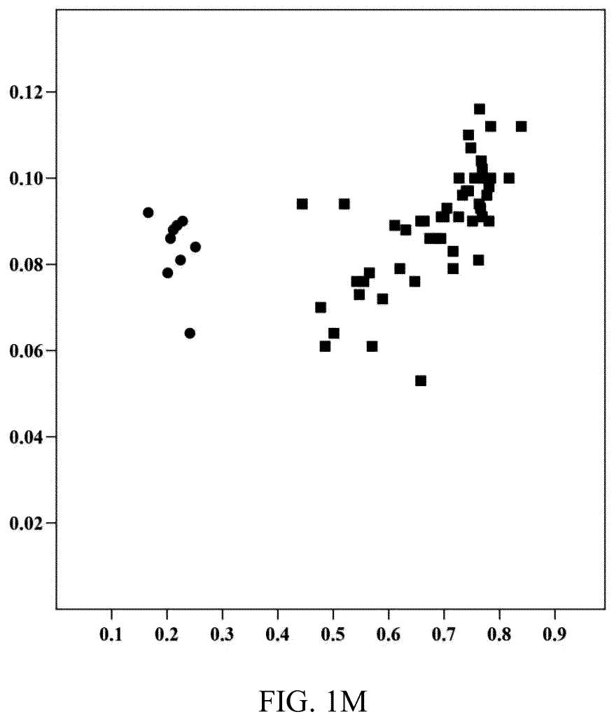

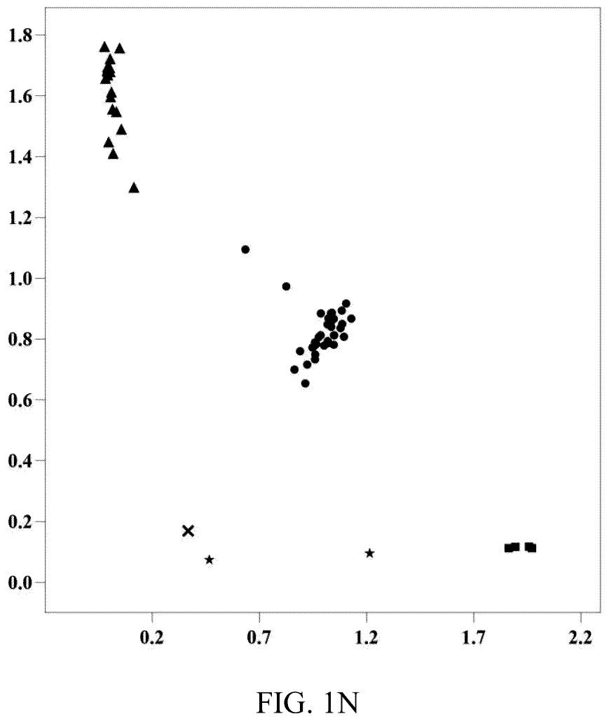

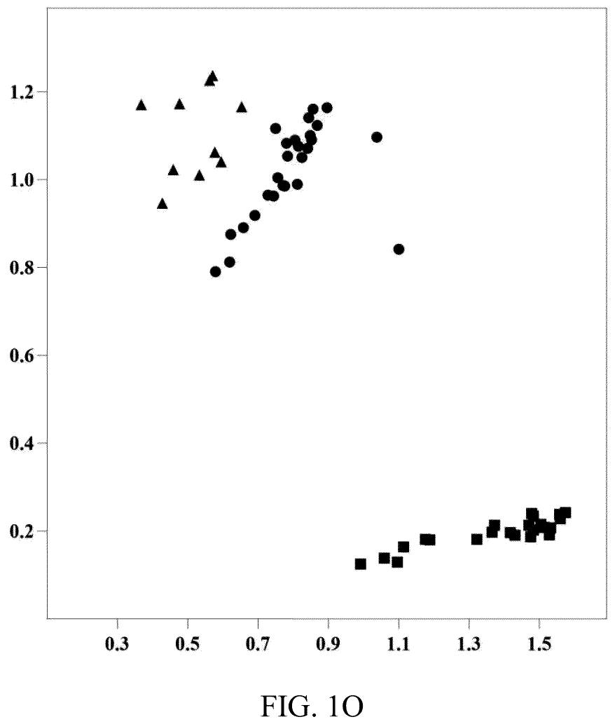

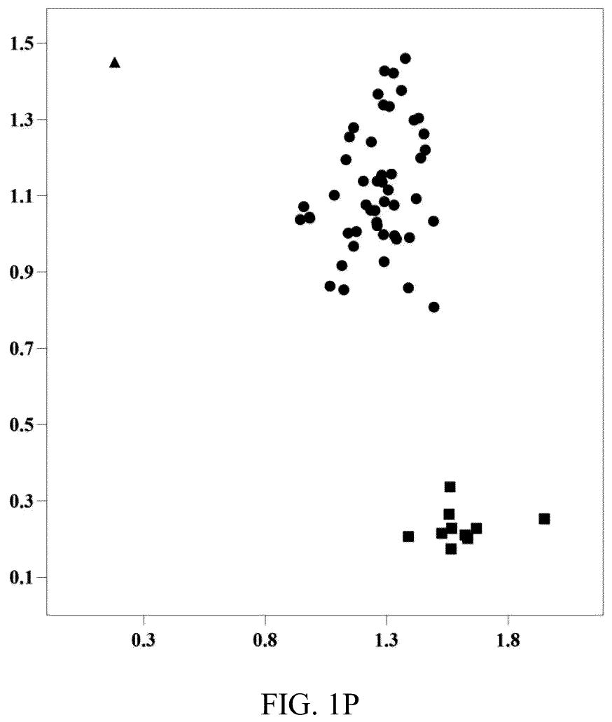

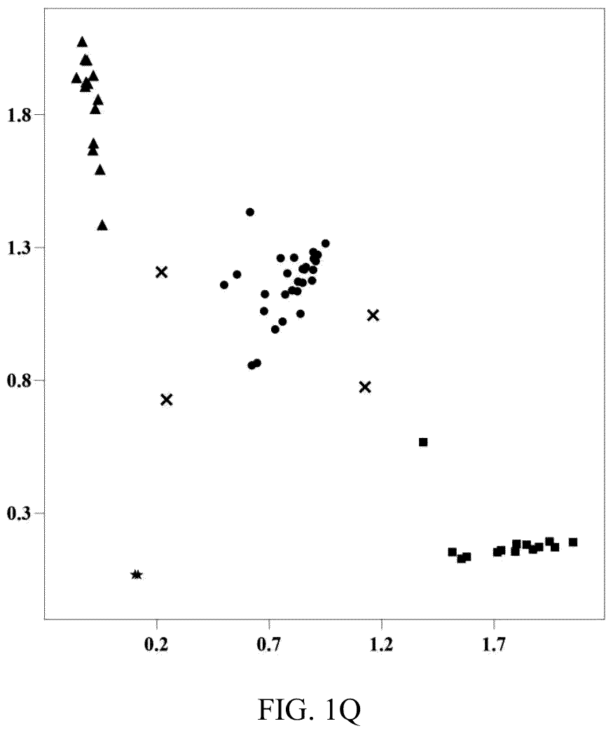

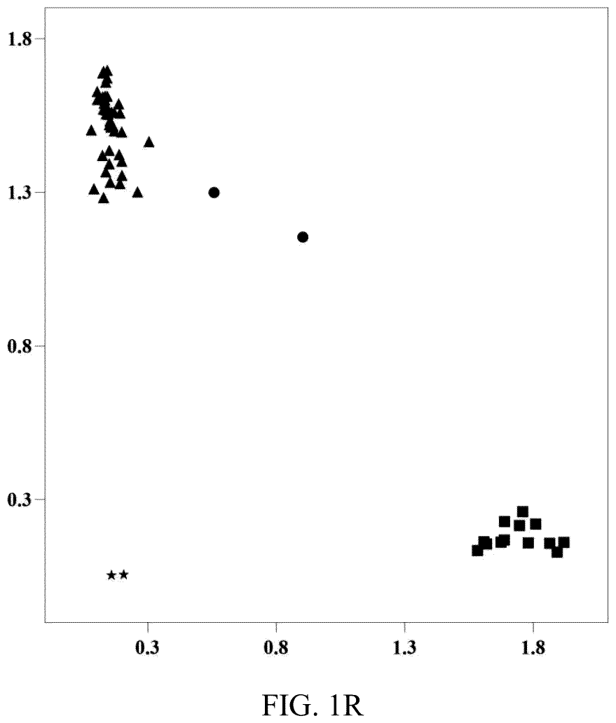

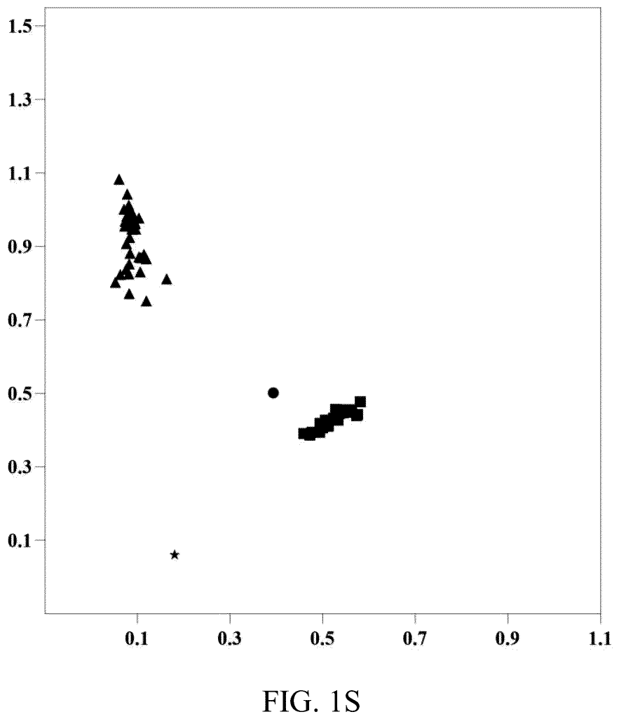

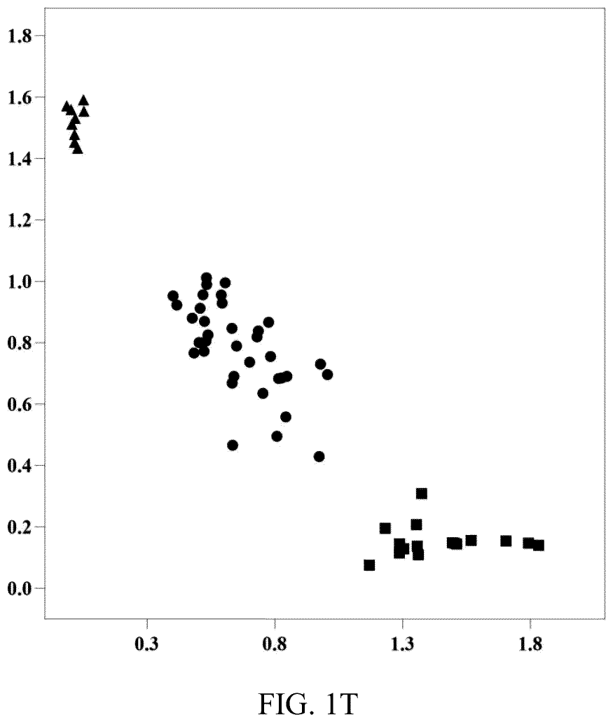

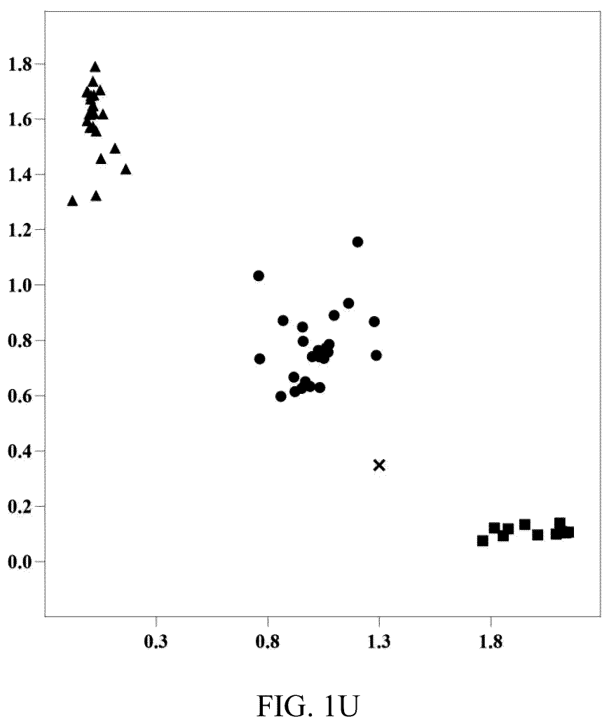

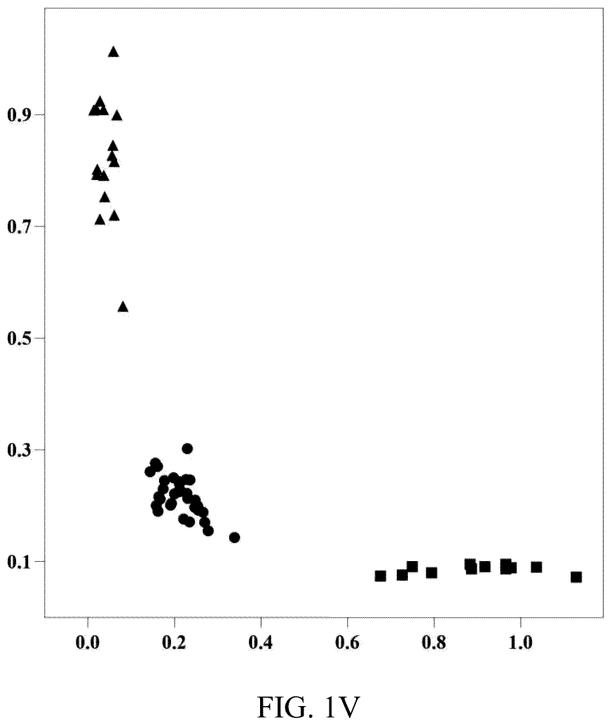

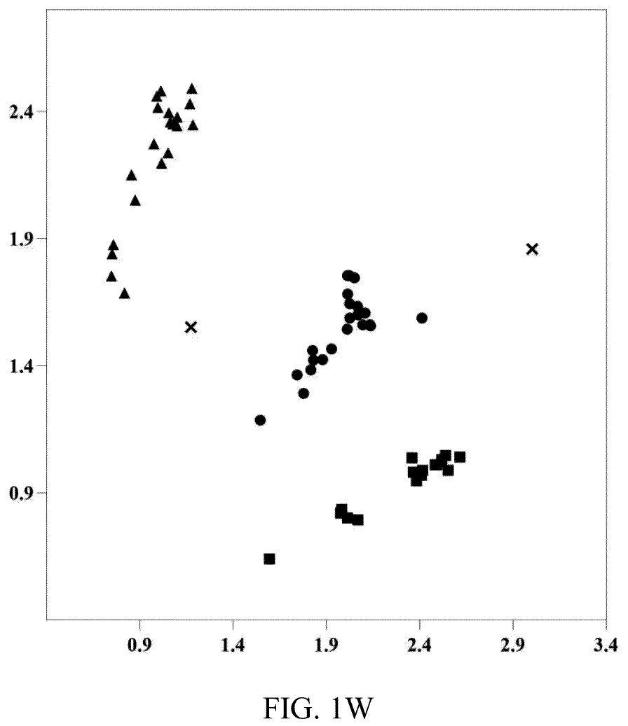

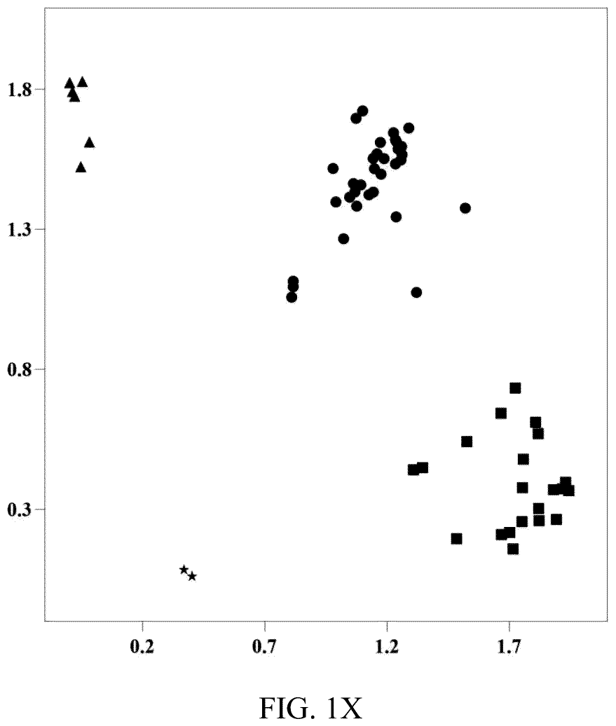

The SNP typing results of some primer sets for Elymus sibiricus germplasm for testing are shown in FIG. 1.

The results showed that the 31 primer sets can obtain good typing results in 60 Elymus sibiricus germplasms for testing. These germplasms were divided into three genotypes. The sample genotypes clustered near the X-axis were genotypes connected to the FAM fluorescent label sequence, and the sample genotypes clustered near the Y-axis were genotypes connected to VIC fluorescent label sequence, both of which were homozygous genotypes. The two fluorescence ratios in the middle area are equal, indicating a heterozygous genotype, and the square near origin point is a negative control NTC, which is always clustered together and close to the base and does not produce a fluorescent signal. It can be seen that primer combination developed in Embodiment 1 can be applied to the identification of Elymus sibiricus germplasm.

### Embodiment 3: Identification of Elymus sibiricus Germplasm to be Tested and its Genetic Relationship with the Standard Elymus sibiricus Germplasm

The present embodiment provides a method for detecting whether the Elymus sibiricus germplasm to be tested belongs to germplasms of 60 Elymus sibiricus for testing and identifying its genetic relationship with the standard Elymus sibiricus germplasm.

1. Extraction of Genomic DNA from Elymus sibiricus Germplasm to be Tested

According to the method of step 1 in the Embodiment 2, the “leaves of Elymus sibiricus germplasm for testing” were replaced with “leaves of Elymus sibiricus germplasm to be tested”, and other steps remained unchanged to obtain the genomic DNA of Elymus sibiricus germplasm to be tested.

### 2. Configuration of SNP Primers and PCR Reaction System

According to the method of step 2 in the Embodiment 2, the “genomic DNA of Elymus sibiricus germplasm for testing” was replaced by “genomic DNA of Elymus sibiricus germplasm to be tested”, and other steps remained unchanged to obtain PCR product of the Elymus sibiricus germplasm to be tested.

### 3. Fluorescence Signal Detection

The PCR products of Elymus sibiricus germplasm to be tested were taken for fluorescence signal analysis, and the genotypes of 31 SNP sites were obtained.

The genotypes of 31 SNP sites of Elymus sibiricus germplasm to be tested were compared with the genotypes of the 31 SNP sites of the 60 Elymus sibiricus germplasm for testing (Table 7-9), and the number of different SNP sites between the Elymus sibiricus germplasm to be tested and 60 standard Elymus sibiricus germplasms was counted, and then the judgments were made as follows:

If the number of difference sites between the Elymus sibiricus germplasm to be tested and a certain standard Elymus sibiricus germplasm is 2 or more, the Elymus sibiricus germplasm to be tested and standard Elymus sibiricus germplasm belong to different Elymus sibiricus germplasm. The more the number of difference sites, the more distant the genetic relationship.

If the number of difference sites between the Elymus sibiricus germplasm to be tested and a certain standard Elymus sibiricus germplasm is 1 or 0, Elymus sibiricus germplasm to be tested and standard Elymus sibiricus germplasm are or are suspected to be the identical Elymus sibiricus germplasm.

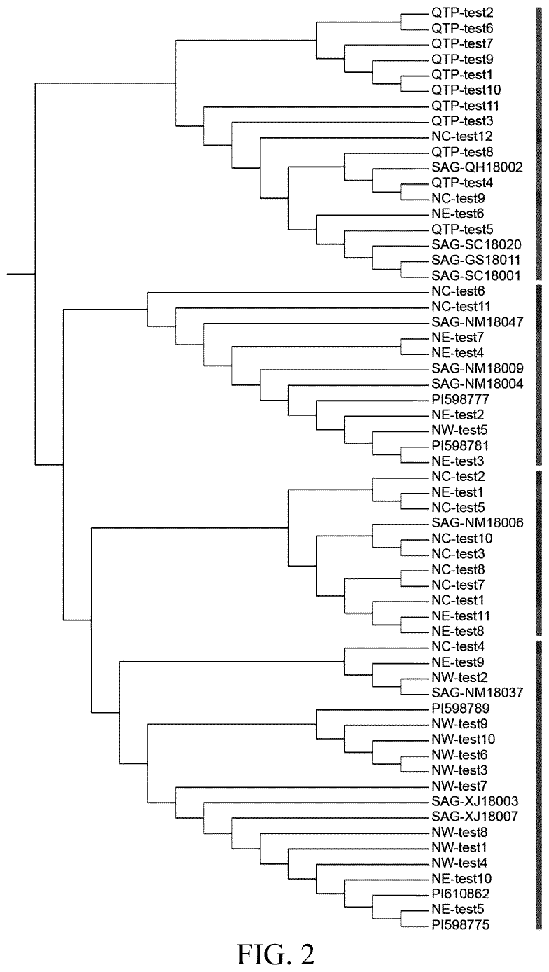

Furthermore, if Elymus sibiricus germplasm to be tested is not any of the standard Elymus sibiricus germplasms in the above-mentioned DNA fingerprint database, the relationship between the Elymus sibiricus germplasm to be tested and standard Elymus sibiricus germplasm can not be identified. The specific method is as follows: According to the genotyping results of the above-mentioned Elymus sibiricus germplasm to be tested and the standard Elymus sibiricus germplasm in the DNA fingerprint database, the distance matrix between the two individuals is calculated based on SNP using MEGA X software. And phylogenetic tree (FIG. 2) is constructed using the neighbor-joining method, and 1000 bootstrap repetitions are performed. The population structure is analyzed using Admixture software (v1.3.0). The relationship between them is identified based on the genetic distance and the clustering results in the phylogenetic tree. When the genetic distance between the Elymus sibiricus germplasm to be tested and a certain standard Elymus sibiricus germplasm is smaller, the closer the relationship is, and they are obviously clustered together in the phylogenetic tree. Conversely, the relationship is farther.

Further, the materials required for research and utilization of Elymus sibiricus germplasm can be screened based on above-mentioned kinship, such as core germplasm construction, breeding population selection, genetic diversity research, etc.

The above specific description is only used to illustrate the present invention, but not limited to the technical solutions described in the embodiments of the present invention. These skilled in the art should understand that the present invention can still be modified or replaced by equivalents to achieve the identical technical effects. As long as the use requirements are met, they are within the protection scope of the present invention.

TABLE 7
DNA Fingerprint Database of Elymus sibiricus Germplasm Resources Based on 31 SNP Loci

| Number of | | NW- | QTP- | NW- | NC- | NW- | NE- | NE- | NC- | SAG- | QTP- | NW- | SAG- |
|---|---|---|---|---|---|---|---|---|---|---|---|---|---|
| samples | | test1 | test6 | test4 | test3 | test7 | test8 | test2 | test9 | XJ18003 | test8 | test5 | NM18037 |
| Number of | KASP-SNP1 | C:G | G:G | C:C | C:C | C:C | C:C | G:G | C:C | C:G | C:G | C:G | C:G |
| KASP- | KASP-SNP2 | T:T | G:G | T:T | T:G | T:T | G:G | T:G | G:G | T:T | T:G | T:T | T:T |
| SNP | KASP-SNP3 | G:G | G:G | G:G | G:T | G:T | G:G | G:G | G:T | —:— | G:T | G:G | G:T |
| | KASP-SNP4 | A:A | A:G | A:A | A:G | A:A | A:G | A:G | A:G | A:A | A:G | A:G | A:A |
| | KASP-SNP5 | A:A | C:A | C:A | A:A | C:A | A:A | A:A | C:A | A:A | A:A | C:A | C:A |
| | KASP-SNP6 | C:C | T:T | C:C | C:C | C:C | C:C | C:C | T:T | C:C | T:T | C:C | C:C |
| | KASP-SNP7 | A:A | G:G | A:A | A:A | A:A | A:A | G:G | A:G | A:A | A:A | G:G | A:A |
| | KASP-SNP8 | G:G | C:C | C:C | C:C | G:G | C:C | C:C | C:C | C:C | C:C | C:C | C:C |
| | KASP-SNP9 | G:A | G:A | G:A | G:G | G:G | G:G | G:A | G:A | A:A | G:A | G:G | G:A |
| | KASP-SNP10 | A:A | A:A | A:A | A:A | A:A | A:A | A:A | A:A | A:A | A:A | A:A | A:A |
| | KASP-SNP11 | C:C | C:C | C:C | C:C | A:A | A:A | C:C | C:C | A:A | C:C | C:C | C:C |
| | KASP-SNP12 | G:G | G:A | G:A | G:A | G:G | G:A | G:G | A:A | G:G | A:A | G:A | G:G |
| | KASP-SNP13 | A:A | A:A | T:T | A:A | A:A | A:A | T:T | A:A | T:T | A:A | T:T | A:A |
| | KASP-SNP14 | A:G | A:G | A:G | A:G | G:G | A:A | A:G | A:A | A:G | A:G | A:A | A:G |
| | KASP-SNP15 | G:G | G:G | G:G | G:G | G:G | G:G | G:A | G:G | G:G | G:G | G:A | G:G |
| | KASP-SNP16 | —:— | G:A | G:G | —:— | A:A | A:A | A:A | A:A | A:A | A:A | A:A | A:A |
| | KASP-SNP17 | T:A | A:A | T:A | T:A | T:T | T:A | A:A | A:A | T:A | A:A | A:A | T:A |
| | KASP-SNP18 | C:C | A:A | A:A | A:A | C:A | C:A | C:C | C:A | A:A | A:A | A:A | A:A |
| | KASP-SNP19 | A:G | G:G | A:G | A:G | A:G | A:G | A:A | A:A | A:G | A:A | A:A | G:G |
| | KASP-SNP20 | A:G | G:G | G:G | A:A | A:A | A:A | A:G | A:A | G:G | G:G | —:— | G:G |
| | KASP-SNP21 | G:A | A:A | G:A | G:A | G:A | G:A | G:G | G:G | G:A | G:G | G:G | A:A |
| | KASP-SNP22 | C:T | T:T | C:C | C:T | C:T | C:T | T:T | C:T | C:T | C:T | C:C | T:T |
| | KASP-SNP23 | A:A | G:G | G:G | A:A | A:A | A:A | G:G | G:A | A:A | A:A | A:A | A:A |
| | KASP-SNP24 | T:T | C:C | T:T | C:C | T:T | C:C | C:C | C:C | T:T | C:C | C:C | T:T |
| | KASP-SNP25 | T:T | T:C | T:T | T:C | T:C | T:C | T:C | T:C | T:T | T:C | T:T | T:C |
| | KASP-SNP26 | C:A | A:A | A:A | C:C | A:A | C:C | C:A | C:A | C:A | A:A | A:A | C:A |
| | KASP-SNP27 | T:T | A:T | T:T | A:T | T:T | A:T | A:T | A:T | T:T | A:A | A:T | T:T |
| | KASP-SNP28 | G:G | G:G | G:G | G:G | G:G | —:— | G:G | —:— | G:G | G:G | —:— | G:G |
| | KASP-SNP29 | T:T | C:C | T:C | T:T | T:C | T:C | T:C | C:C | T:T | C:C | C:C | T:T |
| | KASP-SNP30 | A:C | C:C | A:A | C:C | A:A | A:C | A:C | A:C | A:A | A:C | A:C | A:A |
| | KASP-SNP31 | T:T | T:T | C:C | T:T | T:T | T:C | T:T | T:T | T:C | T:T | T:T | T:T |

| Number of | NW- | NE- | QTP- | NC- | NW- | SAG- | | NC- | NW- | NE- | SAG- |
|---|---|---|---|---|---|---|---|---|---|---|---|
| samples | test8 | test9 | test9 | test10 | test2 | NM18009 | PI598777 | test4 | test9 | test10 | QH18002 |

| | | | | | | | | | | | | |
|---|---|---|---|---|---|---|---|---|---|---|---|---|
| Number of | KASP-SNP1 | C:G | C:G | C:G | C:G | C:G | C:C | C:G | C:G | C:G | C:C | C:C |
| KASP- | KASP-SNP2 | T:T | T:G | T:T | T:T | T:T | G:G | G:G | T:T | T:T | T:G | T:T |
| SNP | KASP-SNP3 | G:T | G:T | G:T | G:T | G:T | G:G | G:G | G:T | G:G | G:G | G:G |
| | KASP-SNP4 | A:A | A:G | A:G | A:A | A:A | A:A | A:G | A:A | A:A | A:A | A:G |
| | KASP-SNP5 | C:C | C:C | C:A | C:A | C:A | A:A | C:A | C:A | A:A | C:A | C:A |
| | KASP-SNP6 | C:C | C:C | T:T | C:C | C:C | C:C | T:T | C:C | C:C | C:C | T:T |
| | KASP-SNP7 | A:A | A:A | G:G | A:A | A:A | A:A | G:G | A:A | A:A | A:A | A:A |
| | KASP-SNP8 | C:C | C:C | C:C | C:C | C:C | G:G | C:C | C:C | C:C | C:C | C:C |
| | KASP-SNP9 | G:A | G:G | G:A | A:A | A:A | G:G | G:A | A:A | A:A | G:G | G:A |
| | KASP-SNP10 | T:A | T:A | A:A | A:A | A:A | T:T | A:A | A:A | A:A | T:A | A:A |
| | KASP-SNP11 | A:A | C:C | C:C | C:C | C:C | C:C | C:C | C:C | A:A | C:C | C:C |
| | KASP-SNP12 | G:G | G:G | G:A | G:A | G:G | G:A | A:A | G:G | G:A | G:G | G:A |
| | KASP-SNP13 | A:A | A:A | T:T | A:A | A:A | T:T | T:T | A:A | A:A | A:A | A:A |
| | KASP-SNP14 | A:A | A:G | A:G | A:G | G:G | A:G | A:A | A:G | A:A | A:A | A:G |
| | KASP-SNP15 | G:G | G:G | G:G | G:G | G:G | G:A | G:G | G:G | G:G | G:G | G:G |
| | KASP-SNP16 | —:— | A:A | —:— | —:— | A:A | —:— | A:A | A:A | A:A | G:G | A:A |
| | KASP-SNP17 | T:T | T:A | A:A | T:A | T:A | A:A | A:A | T:A | T:T | T:A | T:A |
| | KASP-SNP18 | A:A | C:A | C:A | A:A | A:A | A:A | A:A | A:A | C:A | C:A | C:A |
| | KASP-SNP19 | A:G | A:G | G:G | A:G | A:G | A:A | A:A | A:A | A:A | G:G | A:A |
| | KASP-SNP20 | A:A | G:G | A:A | A:A | A:A | G:G | G:G | G:G | G:G | A:A | G:G |
| | KASP-SNP21 | G:A | G:A | A:A | G:A | G:A | G:G | G:G | G:A | G:A | A:A | G:G |
| | KASP-SNP22 | C:T | C:T | C:C | T:T | T:T | C:C | T:T | C:T | C:T | C:C | C:C |
| | KASP-SNP23 | A:A | A:A | G:G | A:A | G:G | A:A | A:A | A:A | A:A | A:A | A:A |
| | KASP-SNP24 | T:T | T:T | C:C | C:C | T:T | C:C | C:C | T:T | T:T | T:T | C:C |
| | KASP-SNP25 | T:T | T:C | T:C | T:C | T:C | T:C | T:C | T:C | T:C | T:C | C:C |
| | KASP-SNP26 | A:A | C:A | C:A | C:C | C:A | C:C | A:A | C:A | A:A | A:A | A:A |
| | KASP-SNP27 | A:T | T:T | A:T | A:T | T:T | A:T | A:A | T:T | T:T | A:T | A:A |
| | KASP-SNP28 | G:G | —:— | G:G | G:G | G:G | —:— | G:G | G:A | G:G | G:G | —:— |
| | KASP-SNP29 | T:C | T:T | C:C | T:T | T:C | T:T | C:C | C:C | C:C | T:T | C:C |
| | KASP-SNP30 | A:C | C:C | C:C | C:C | A:A | A:A | A:C | A:C | A:C | A:A | A:C |
| | KASP-SNP31 | T:T | C:C | T:T | T:T | T:T | T:T | T:T | T:T | T:T | T:T | T:T |

Mean: —:— Signal absence at the corresponding SNP locus for the listed germplasm.

TABLE 8
DNA Fingerprint Database of Elymus sibiricus Germplasm Resources Based on 31 SNP Loci

| Number of | | NC- | SAG- | NE- | QTP- | NC- | NW- | NE- | QTP- | NC- | NW- | NE- | QTP- | NC- | |
|---|---|---|---|---|---|---|---|---|---|---|---|---|---|---|---|
| samples | | test11 | XJ18007 | test3 | test4 | test5 | test10 | test11 | test10 | test12 | test3 | test4 | test5 | test6 | PI610862 |
| Number of | KASP-SNP1 | C:G | C:G | C:G | C:C | C:G | G:G | C:C | C:G | C:G | C:G | C:C | G:G | C:G | C:G |
| KASP- | KASP-SNP2 | T:G | T:G | T:G | G:G | T:T | T:T | T:G | G:G | T:G | T:T | G:G | T:T | T:G | T:T |
| SNP | KASP-SNP3 | G:T | G:T | G:G | G:T | G:G | G:G | G:G | G:T | G:T | —:— | G:G | G:T | G:T | G:T |
| | KASP-SNP4 | A:G | A:A | A:G | A:G | A:A | G:G | A:A | A:G | A:G | G:G | A:G | A:A | A:G | A:A |
| | KASP-SNP5 | C:A | A:A | A:A | C:A | C:A | C:A | C:A | C:A | C:A | C:A | A:A | C:A | C:A | C:A |
| | KASP-SNP6 | C:C | C:C | C:T | T:T | C:C | C:C | C:C | T:T | T:T | C:C | C:C | T:T | C:C | C:C |
| | KASP-SNP7 | A:A | A:A | A:G | A:A | A:A | A:A | A:A | A:G | A:A | A:A | A:A | A:A | A:A | G:G |
| | KASP-SNP8 | C:C | C:C | C:G | C:C | C:C | C:C | C:C | C:C | C:C | G:G | C:C | C:C | C:C | C:C |
| | KASP-SNP9 | A:A | A:A | G:A | G:A | G:A | A:A | G:G | G:A | G:A | A:A | G:A | G:A | G:A | G:G |
| | KASP-SNP10 | A:A | A:A | A:A | A:A | A:A | A:A | A:A | A:A | A:A | A:A | A:A | A:A | A:A | A:A |
| | KASP-SNP11 | C:C | A:A | C:C | C:C | C:C | A:A | C:C | C:C | C:C | A:A | C:C | C:C | C:C | C:A |
| | KASP-SNP12 | G:A | G:A | A:A | A:A | G:A | A:A | G:A | G:A | G:A | G:A | G:G | G:A | G:G | G:G |
| | KASP-SNP13 | T:T | A:A | T:T | T:T | A:A | A:A | A:A | A:T | A:A | A:A | T:T | T:T | A:A | T:T |
| | KASP-SNP14 | A:G | A:G | A:G | A:A | A:G | A:A | A:A | A:G | A:A | A:A | A:G | A:A | A:G | A:A |
| | KASP-SNP15 | G:G | G:G | G:G | G:G | G:G | G:G | G:G | G:G | G:G | G:G | G:G | G:A | G:G | G:G |
| | KASP-SNP16 | A:A | —:— | A:A | A:A | A:A | A:A | A:A | G:A | —:— | G:A | —:— | A:A | G:G | G:G |
| | KASP-SNP17 | T:A | T:A | A:A | A:A | T:A | T:T | T:A | T:A | A:A | T:A | T:A | A:A | T:A | T:A |
| | KASP-SNP18 | C:C | A:A | A:A | A:A | A:A | A:A | C:C | C:A | C:A | A:A | —:— | —:— | —:— | A:A |
| | KASP-SNP19 | A:G | A:G | A:A | A:A | G:G | A:G | A:G | A:G | A:A | A:A | A:A | A:A | A:G | A:G |
| | KASP-SNP20 | A:A | G:G | G:G | A:G | G:G | A:A | A:G | A:A | G:G | G:G | G:G | A:A | G:G | A:A |
| | KASP-SNP21 | G:A | G:A | G:G | G:G | A:A | G:A | G:A | G:A | G:G | G:G | G:G | G:G | G:A | G:A |
| | KASP-SNP22 | C:T | C:T | C:T | C:T | C:T | C:T | C:T | —:— | T:T | C:C | C:T | C:C | C:T | C:T |
| | KASP-SNP23 | A:A | A:A | A:A | A:A | A:A | A:A | A:A | G:G | A:A | A:A | A:A | A:A | A:A | A:A |
| | KASP-SNP24 | T:T | T:T | T:C | C:C | C:C | T:T | C:C | C:C | C:C | T:T | T:T | C:C | C:C | C:C |
| | KASP-SNP25 | T:C | T:T | T:C | C:C | T:T | T:T | T:C | T:T | C:C | T:T | T:C | C:C | T:T | T:C |
| | KASP-SNP26 | C:A | C:A | C:A | A:A | C:C | A:A | C:A | C:A | A:A | C:A | A:A | A:A | A:A | C:C |
| | KASP-SNP27 | T:T | T:T | A:T | A:A | A:T | A:T | A:T | A:T | A:T | T:T | A:A | A:T | A:T | A:T |
| | KASP-SNP28 | G:G | G:G | G:A | —:— | —:— | —:— | —:— | G:G | G:G | G:G | —:— | G:G | G:G | G:G |
| | KASP-SNP29 | T:C | T:T | T:C | C:C | T:T | T:C | T:C | T:C | T:C | C:C | C:C | C:C | C:C | T:T |
| | KASP-SNP30 | A:C | A:A | A:C | A:C | A:C | A:C | A:C | A:C | A:C | A:A | A:A | A:A | A:A | A:A |
| | KASP-SNP31 | T:T | T:T | T:T | T:T | T:T | T:T | C:C | T:T | T:T | T:C | C:C | T:T | T:T | C:C |

| Number of | | | QTP- | QTP- | NE- | SAG- | QTP- | | QTP- | SAG- | SAG- |
|---|---|---|---|---|---|---|---|---|---|---|---|
| samples | | PI598775 | test11 | test2 | test5 | SC18020 | test1 | PI598789 | test3 | NM18006 | SC18001 |
| Number of | KASP-SNP1 | C:C | C:G | C:C | C:G | G:G | C:G | C:G | G:G | C:G | G:G |
| KASP- | KASP-SNP2 | T:G | T:T | G:G | T:T | T:T | G:G | T:G | G:G | T:T | T:G |
| SNP | KASP-SNP3 | —:— | G:T | G:G | G:T | G:T | G:T | G:G | —:— | G:T | G:T |
| | KASP-SNP4 | A:A | A:G | A:A | A:A | A:G | A:G | A:A | A:G | A:A | A:G |
| | KASP-SNP5 | C:A | C:A | C:A | C:A | C:A | C:A | C:A | C:A | C:A | C:A |
| | KASP-SNP6 | C:C | T:T | T:T | C:C | T:T | C:C | C:C | T:T | C:C | T:T |
| | KASP-SNP7 | G:G | A:A | —:— | G:G | A:A | A:A | A:A | A:A | A:A | A:G |
| | KASP-SNP8 | G:G | C:C | C:C | G:G | G:G | C:C | C:C | C:C | C:C | C:C |
| | KASP-SNP9 | G:A | G:A | G:A | G:A | G:A | G:A | G:A | G:A | G:A | G:A |
| | KASP-SNP10 | T:A | A:A | A:A | T:A | A:A | A:A | A:A | A:A | A:A | A:A |
| | KASP-SNP11 | C:C | A:A | C:C | C:C | C:A | C:C | A:A | A:A | C:C | C:C |
| | KASP-SNP12 | G:G | G:A | A:A | G:A | G:G | A:A | G:G | A:A | G:G | G:A |
| | KASP-SNP13 | T:T | A:A | A:T | A:A | A:A | A:A | A:A | T:T | A:A | A:T |
| | KASP-SNP14 | A:G | A:G | A:G | A:G | A:G | A:A | A:G | A:A | A:G | A:A |
| | KASP-SNP15 | G:A | G:G | G:G | G:G | G:A | G:G | G:G | G:G | G:G | G:G |
| | KASP-SNP16 | G:G | A:A | G:A | —:— | A:A | G:A | A:A | —:— | G:G | A:A |
| | KASP-SNP17 | A:A | T:A | A:A | T:A | T:A | T:A | T:A | A:A | T:A | A:A |
| | KASP-SNP18 | C:C | C:A | C:A | —:— | C:C | C:C | C:A | C:C | C:A | C:A |
| | KASP-SNP19 | G:G | G:G | G:G | G:G | A:G | A:G | A:G | A:A | A:A | A:A |
| | KASP-SNP20 | A:G | G:G | A:A | G:G | A:G | A:A | G:G | A:A | A:A | A:G |
| | KASP-SNP21 | A:A | A:A | A:A | A:A | G:A | G:A | G:A | G:G | G:G | G:G |
| | KASP-SNP22 | C:C | T:T | T:T | C:C | T:T | C:T | C:T | —:— | C:T | C:C |
| | KASP-SNP23 | A:A | A:A | G:G | A:A | G:G | G:G | A:A | A:A | G:A | G:G |
| | KASP-SNP24 | C:C | C:C | C:C | T:T | C:C | C:C | T:T | C:C | C:C | C:C |
| | KASP-SNP25 | T:C | T:T | C:C | C:C | T:C | T:C | C:C | C:C | C:C | T:C |
| | KASP-SNP26 | C:A | A:A | A:A | C:A | A:A | C:C | C:A | A:A | C:A | A:A |
| | KASP-SNP27 | A:T | A:A | A:A | A:T | A:T | A:T | A:T | A:A | A:T | A:T |
| | KASP-SNP28 | G:G | G:G | —:— | G:A | G:G | G:G | G:G | G:G | G:G | G:A |
| | KASP-SNP29 | T:T | T:C | T:C | C:C | C:C | C:C | C:C | —:— | T:T | T:C |
| | KASP-SNP30 | A:C | A:C | A:C | A:A | A:C | C:C | A:C | A:C | A:A | A:C |
| | KASP-SNP31 | T:C | T:T | T:T | C:C | C:C | T:T | C:C | T:T | T:T | T:C |

Mean: —:— Signal absence at the corresponding SNP locus for the listed germplasm.

TABLE 9
DNA Fingerprint Database of Elymus sibiricus Germplasm Resources Based on 31 SNP Loci

| Number of | | NE- | SAG- | NC- | | SAG- | NC- | NW- | NE- | QTP- | NC- | NE- | SAG- | NC- |
|---|---|---|---|---|---|---|---|---|---|---|---|---|---|---|
| samples | | test6 | NM18004 | test7 | PI598781 | NM18047 | test1 | test6 | test7 | test7 | test8 | test1 | GS18011 | test2 |
| Number of | KASP-SNP1 | G:G | C:G | C:G | G:G | C:C | C:C | C:G | C:C | G:G | C:G | C:G | C:G | C:G |
| KASP-SNP | KASP-SNP2 | T:T | G:G | G:G | T:G | G:G | T:G | T:T | G:G | T:G | G:G | T:G | T:T | T:G |
| | KASP-SNP3 | G:T | G:G | G:G | G:G | G:T | G:G | G:T | G:G | G:T | G:G | G:G | G:G | G:T |
| | KASP-SNP4 | A:G | A:G | A:A | A:G | A:G | A:A | G:G | A:G | A:G | A:G | A:A | A:G | A:A |
| | KASP-SNP5 | C:A | C:A | C:C | C:A | C:A | C:A | C:A | A:A | C:A | C:A | C:A | C:A | C:A |
| | KASP-SNP6 | T:T | T:T | C:C | C:C | C:C | C:C | C:C | T:T | C:T | C:C | C:C | —:— | C:C |
| | KASP-SNP7 | A:A | A:A | A:A | G:G | A:A | A:A | A:A | A:A | A:G | A:A | A:A | G:G | A:A |
| | KASP-SNP8 | C:C | C:C | C:C | G:G | C:C | C:C | G:G | C:C | C:G | C:C | C:C | C:C | C:C |
| | KASP-SNP9 | G:A | A:A | G:A | G:A | G:A | G:A | A:A | G:A | G:A | A:A | A:A | G:G | G:A |
| | KASP-SNP10 | A:A | A:A | A:A | A:A | A:A | T:A | A:A | A:A | A:A | A:A | A:A | A:A | —:— |
| | KASP-SNP11 | C:C | C:C | C:C | C:A | C:C | C:C | A:A | C:C | C:C | C:C | C:C | C:C | C:C |
| | KASP-SNP12 | G:A | G:A | G:G | A:A | G:G | G:G | G:A | G:G | G:A | G:A | G:A | G:A | G:G |
| | KASP-SNP13 | T:T | T:T | A:A | T:T | T:T | A:A | A:A | A:T | A:T | A:A | A:A | T:T | A:A |
| | KASP-SNP14 | A:A | A:A | A:G | A:A | A:G | A:G | A:A | —:— | A:A | A:A | A:A | A:G | —:— |
| | KASP-SNP15 | G:G | G:A | G:G | G:A | G:G | G:G | G:G | G:G | G:G | G:G | G:G | G:A | G:G |
| | KASP-SNP16 | —:— | G:G | G:A | A:A | A:A | G:A | —:— | G:A | G:A | A:A | A:A | G:A | A:A |
| | KASP-SNP17 | A:A | T:A | T:A | A:A | T:A | T:A | T:A | T:A | A:A | T:A | T:A | T:A | —:— |
| | KASP-SNP18 | —:— | C:A | C:A | C:A | C:A | C:A | C:A | C:C | C:A | C:C | C:C | C:A | C:C |
| | KASP-SNP19 | A:A | A:A | A:G | A:A | A:G | A:G | A:A | A:A | A:A | A:G | A:G | A:A | G:G |
| | KASP-SNP20 | G:G | A:A | A:A | A:A | A:G | A:A | G:G | A:G | A:G | A:G | A:G | A:A | A:A |
| | KASP-SNP21 | G:G | G:G | G:A | G:G | G:A | G:A | G:G | G:G | G:G | G:A | G:A | G:G | A:A |
| | KASP-SNP22 | T:T | T:T | C:T | T:T | C:T | C:T | C:C | —:— | —:— | T:T | C:C | C:C | C:T |
| | KASP-SNP23 | A:A | A:A | A:A | A:A | A:A | A:A | A:A | A:A | G:G | A:A | G:G | G:G | A:A |
| | KASP-SNP24 | C:C | C:C | C:C | T:T | C:C | C:C | T:T | C:C | C:C | C:C | T:T | C:C | C:C |
| | KASP-SNP25 | T:C | T:T | T:C | T:C | T:C | T:C | T:T | T:C | T:C | T:C | T:T | T:T | T:C |
| | KASP-SNP26 | A:A | C:C | A:A | C:A | C:C | C:A | C:A | —:— | C:A | C:A | C:C | A:A | C:C |
| | KASP-SNP27 | A:A | A:T | A:T | A:T | A:A | T:T | T:T | A:T | A:T | T:T | A:A | A:A | T:T |
| | KASP-SNP28 | —:— | G:G | G:G | A:A | G:G | —:— | G:A | —:— | G:G | —:— | A:A | G:G | G:A |
| | KASP-SNP29 | C:C | T:C | T:T | T:C | T:C | T:T | C:C | —:— | T:C | T:T | T:C | T:C | T:C |
| | KASP-SNP30 | A:A | A:A | A:A | A:C | A:A | A:C | A:A | A:C | A:C | A:C | A:C | A:A | A:C |
| | KASP-SNP31 | T:T | T:T | T:T | T:T | T:T | T:T | C:C | C:C | T:T | T:T | T:T | C:C | T:T |

mean: —:— Signal absence at the corresponding SNP locus for the listed germplasm.

1. A primer combination for KASP markers used in identification of Elymus sibiricus germplasm resources, wherein nucleotide sequences of the primer combination are shown as SEQ ID NOs: 1-93, wherein the primer combination is used to amplify 31 SNP sites, and wherein the basic information of the 31 SNP sites is shown in the following table:
Basic information of 31 SNP sites

| | | Physical location | Base type | |
|---|---|---|---|---|
| SNP site name | Chromosome | on chromosome | Ref | Alt |
| Es_SNP01 | Es1H | 151369718 | C | G |
| Es_SNP02 | Es1H | 368738668 | T | G |
| Es_SNP03 | Es1St | 4252112 | G | T |
| Es_SNP04 | Es1St | 4254262 | A | G |
| Es_SNP05 | Es2H | 27018962 | C | A |
| Es_SNP06 | Es2H | 189406968 | C | T |
| Es_SNP07 | Es2H | 332856077 | A | G |
| Es_SNP08 | Es2H | 442576323 | C | G |
| Es_SNP09 | Es2St | 373302575 | G | A |
| Es_SNP10 | Es3H | 18510385 | T | A |
| Es_SNP11 | Es3H | 44159550 | C | A |
| Es_SNP12 | Es3St | 68726104 | G | A |
| Es_SNP13 | Es3St | 193423410 | A | T |
| Es_SNP14 | Es4H | 1322297 | A | G |
| Es_SNP15 | Es4H | 56665546 | G | A |
| Es_SNP16 | Es4H | 147223205 | G | A |
| Es_SNP17 | Es4H | 203852028 | T | A |
| Es_SNP18 | Es4St | 8175058 | C | A |
| Es_SNP19 | Es4St | 359198064 | A | G |
| Es_SNP20 | Es5H | 76547989 | A | G |
| Es_SNP21 | Es5St | 67286670 | G | A |
| Es_SNP22 | Es5St | 108042483 | C | T |
| Es_SNP23 | Es5St | 429659992 | G | A |
| Es_SNP24 | Es6St | 306971115 | T | C |
| Es_SNP25 | Es6St | 381408141 | T | C |
| Es_SNP26 | Es7H | 46465420 | C | A |
| Es_SNP27 | Es7H | 131252857 | A | T |
| Es_SNP28 | Es7H | 312478825 | G | A |
| Es_SNP29 | Es7H | 436380001 | T | C |
| Es_SNP30 | Es7St | 20075622 | A | C |
| Es_SNP31 | Es7St | 122289419 | T | C |

2. The primer combination according to claim 1, wherein the 5′ ends of the specific regions of the first forward primer and the second forward primer in each primer set are respectively linked to the different universal fluorescent tag sequences.

3. (canceled)

4. A method for the high-throughput identification of Elymus sibiricus germplasm, wherein the method comprises the following steps:
1) extracting genomic DNA from the Elymus sibiricus sample to be tested,
2) using the genomic DNA obtained in step 1) as a template to perform the KASP using the primer combination according to claim 1, thereby obtaining amplification products,
3) analyzing fluorescence signal of the amplified product, and determining the genotype of the 1st SNP site to the 31th SNP site in the Elymus sibiricus genome to be tested based on the fluorescence signal of the amplified product obtained by each KASP-SNP primer set, and
4) comparing genotype results at SNP sites 1 to 31 of Elymus sibiricus genome to be tested obtained in step 3) with genome of known Elymus sibiricus cultivar, wherein it is determined that Elymus sibiricus germplasm to be tested does not belong to the known Elymus sibiricus cultivar, if the number of differing SNP sites between Elymus sibiricus germplasm to be tested and the known Elymus sibiricus cultivar is two or more, and wherein it is determined that the Elymus sibiricus germplasm to be tested belongs to the known Elymus sibiricus cultivar or is suspected to be the known Elymus sibiricus cultivar if the number of the differing SNP sites is zero or one.
